(19) United States
(12) Patent Application Publication
(10) Pub. No.: US 2012/0065439 A1
(43) Pub. Date: Mar. 15, 2012

(54) SYSTEM FOR PHOTOBIOSYNTHETIC PRODUCTION, SEPARATION AND SATURATION OF CARBONACEOUS CHEMICALS AND FUELS

(75) Inventors: Christian Siemer, Chadds Ford, PA (US); Simon Eric Aspland, Baltimore, MD (US); Annastasiah Mudiwa Mhaka, Baltimore, MD (US); Philip Goelet, Reisterstown, MD (US)

(73) Assignee: ZUVACHEM, INC.

(21) Appl. No.: 13/203,717

(22) PCT Filed: Mar. 1, 2010
(86) PCT No.: PCT/US10/25816

§ 371 Date: Dec. 1, 2011

Related U.S. Application Data
(60) Provisional application No. 61/156,347, filed on Feb. 27, 2009.
(60) Provisional application No. 61/242,654, filed on Sep. 15, 2009.
(60) Provisional application No. 61/291,817, filed on Dec. 31, 2009.

(51) Int. Cl. C07C 11/10; C12M 1/42; C07C 9/18; C12P 5/02

(52) U.S. Cl. 585/16; 435/167; 435/292.1

(57) ABSTRACT

The present invention provides new energy solutions that are sustainable both environmentally and economically. The invention relates to photo-biocatalytic (PBC) methods and systems designed to produce and isolate carbonaceous chemicals using carbon dioxide, sunlight, and genetically engineered photosynthetic microorganisms. The PBC system comprises of procedural, mechanical and biological components designed for the production of carbonaceous chemicals. In an exemplary embodiment, the system includes a photo-biochemical reactor designed to maintain the genetically modified photosynthetic microorganisms in the optimal condition to capture carbon dioxide and convert it into metabolic intermediates using energy from sunlight, convert the metabolic intermediates into isoprene using recombinant enzymes, allow for the release of isoprene from cells, capture, separate and concentrate isoprene, and ultimately collect the isoprene at levels and in a form that would serve as a viable alternative to petroleum-dependent energy.

### FIELD OF THE INVENTION

The invention relates, in part, to low cost production of renewable carbonaceous chemicals and fuels by a novel system that integrates photosynthetic biological catalysts with mechanical components and chemical separation and catalytic procedures without the need to harvest the biological catalyst or exploit energy in separating said carbonaceous product from the biological catalyst. The invention also relates to a novel system integrating novel biochemical catalysts with mechanical components and procedures for environmentally sustainable energy production.

### BACKGROUND OF INVENTION

Meeting future global demand for energy, fuel, and raw materials in ways that are both economically sound and environmentally benign is, arguably, one of the greatest challenges of our age. During the last century, both the energy and the chemical industries would have been inconceivable without hydrocarbon fossil fuels: oil, gas and coal. The industrialization of China, India and other developing countries is further increasing global demand for transportation fuel, petrochemical products, and grid energy. As a result, the costs of these commodities have climbed significantly. Moreover, the recognition that oil reserves are rapidly declining, that the combustion of fossil fuel is responsible for global warming, and that some of the richest sources of fossil fuels are in politically unstable regions, has heightened the desire for renewable sources of energy, transportation fuel, and industrial chemicals.

One sustainable approach to meet the demand for renewable sources of energy and industrial chemicals currently extracted from petrochemicals is to exploit photosynthesis: nature's method of harnessing solar energy and using carbon dioxide and water to synthesize hydrocarbon-based molecules, the fuel and building blocks of life. In order to do this, it is necessary to create metabolically engineered photosynthetic organisms that can generate renewable chemicals and energy sources. The compounds produced are chemically identical to the ones produced from petroleum, coal, natural gas, etc. but, unlike the case of fossil fuel-derived chemicals, the carbonaceous chemicals of the present invention are produced in a sustainable manner.

Hydrocarbons are any molecules that just contain hydrogen and carbon that can be burnt (oxidized) to form water (H₂O) or carbon dioxide (CO₂). If the combustion is not complete, carbon monoxide (CO) may be formed. As CO can be burnt to produce CO₂, it is also a fuel. More than 500 hydrocarbons are commonly used in liquid fuels. Typically, the molecular weight of the hydrocarbon determines the type of fuel it is best suited for. Gasoline typically contains hydrocarbons with 3 to 12 carbon atoms per molecule, jet fuel typically contains hydrocarbons with 8 and 16 carbon atoms per molecule and diesel typically between 8 and 21 carbon atoms per molecule.

Photosynthesis is a complex biochemical process by which plants, algae and other microorganisms convert light energy into chemical energy to drive the transformation of carbon dioxide and water to cell components. Cyanobacteria (formerly known as blue-green algae) are not eukaryotic alga, but rather are prokaryotic in nature. They represent the oldest fossil accredited with creating atmospheric oxygen and being the evolutionary precursors to chloroplasts, contributing to plants' origin. Like plant cells, cyanobacteria are phototrophic or photoautotrophic (from the Greek “photo” (light), “auto” (self), “troph” (nourishment)), but unlike plant cells, most cyanobacteria do not possess a cellulose cell wall. Cyanobacteria can use nitrate or ammonia as a source of nitrogen, and require phosphorus and micronutrients, such as iron. A number of cyanobacteria have been characterized in the laboratory setting and it has been demonstrated that they can be manipulated genetically.

New genes can be added either on extra-chromosomal plasmids or by integrating them into the cyanobacterial chromosome to confer additional functionality. Plants contain some biochemical pathways that utilize enzymes, for which no equivalents exist in cyanobacteria. These pathways produce a wide diversity of biochemicals. The genes encoding these plant enzymes can be functionally transferred to cyanobacteria by integrating into the genomes of cyanobacteria under the control of a variety of regulatory elements or promoters designed to function constitutively or to be inducible in cyanobacteria. This way, plant biochemical pathways can be reconstituted in genetically engineered cyanobacteria, generating cyanobacteria that produce biochemicals or increased levels of biochemicals they previously made in other ways.

Cyanobacterial genes can also be selectively knocked out using homologous recombination. DNA constructs containing sequences complementary to sequences in the cyanobacterial genome can be directed to specific regions of the genome and can recombine, exchanging a endogenous cyanobacterial gene with a selectable sequence of the DNA construct. This way, endogenous genes that would reduce the production of a desired compound in the cyanobacterium can be selectively deleted.

Combining the addition of exogenous genes from other species and the removal of endogenous cyanobacterial genes, cyanobacteria can be genetically engineered to produce high levels of volatile immiscible olefins such as isoprene and other specific hydrocarbons. The cyanobacteria genetically engineered to produce volatile immiscible olefins and other specific hydrocarbons from carbon dioxide using the power of sunlight are referred to herein as photobiocatalysts.

In the United States, isoprene is produced largely from the by-product C5 hydrocarbon streams of various olefin feed stocks (particularly ethylene) or it can be produced commercially via on-purpose synthetic routes, which were predominant prior to the mid-70's. All current methods of commercial production of isoprene depend upon petroleum as the feedstock, so isoprene shares the price volatility, availability, political unrest and environmental concerns of petroleum. In addition, isoprene production is energy intensive and emits CO₂. Finally, in most instances isoprene contains other carbon-based impurities, requiring further refinement or limited use in high-end applications that require pure cis-isoprene.

In 2007, approximately 800,000 metric tons (>$1B market) of high purity isoprene were produced worldwide, with production almost equalling consumption leading to a tight margin between supply and demand. Globally, over 90% of high purity isoprene was converted to synthetic rubber and thermoplastic elastomers (largely Styrene-Isoprene-Styrene (SIS) Block Copolymers). Isoprene also finds utility in the production of specialty items such as vitamins, pesticides, pharmaceuticals, flavours and epoxy hardeners. A new host of technologies, thermoplastic products and applications, and chemical derivatives are under development. These activities demonstrate the incredible utility of isoprene, especially the high purity isoprene that is substantially free of carbon impurities and made using an environmentally benign process that consumes CO₂ and utilizes solar energy and photobiocatalysts.

There is a need for new sustainable methods and systems to meet the global demands for energy without reliance on petroleum-derived fuel.

### SUMMARY OF THE INVENTION

The present invention provides a method of preparing a carbonaceous chemical. The method includes, (a) maintaining a photobiocatalyst under a culture condition sufficient for said photobiocatalyst to convert a feedstock comprising carbon dioxide into said carbonaceous chemical through photosynthesis. During at least a portion of the period during which the carbonaceous material is being formed, the photobiocatalyst is maintained in a culture medium in contact with an absorbent medium essentially miscible with said carbonaceous chemical.

In various embodiments, the mixture of the carbonaceous chemical and absorbent is separated from the photobiocatalyst. In exemplary embodiments, the carbonaceuous chemical is separated from the absorbent. In various embodiments, the separation of the carbonaceous chemical from the absorbent provides the carbonaceous chemical in an essentially pure state. In an exemplary embodiment, the carbonaceous chemical is produced in essentially pure state with no more purification necessary than separation of the carbonaceous chemical from the absorbent.

In various embodiments of the invention, the photobiocatalyst is cultured under a first set of conditions before it begins to catalyze the carbon dioxide containing feedstock into the carbonaceous compound. In these embodiments, the first culture condition can be the same as or different from the culture conditions under which the photobiocatalyst catalyzes the synthesis of the carbonaceous chemical.

In an exemplary embodiment, the carbonaceous chemical is a terpene. An exemplary terpene prepared by the methods of the invention is a hemiterpene. An exemplary hemiterpene prepared by the method of the invention is isoprene.

In various embodiments of the invention, the absorbent is an organic absorbent, such as a solvent, a wax or an oil. In an exemplary embodiment, the organic absorbent is a hydrocarbon, e.g., a paraffinic or isoparaffinic material. Exemplary isoparaffinic materials of use in the invention include, without limitation, ISOPAR™ (Exxon Mobil), e.g., ISOPAR™. In some embodiments, the system of the invention employs one or more batch or flow-through reactors for the addition of fresh photobiocatalyst and/or removal of spent photobiocatalyst. As those of skill in the art would recognize, one or more components of the system may operate in batch, semi-batch or continuous mode for the addition and removal of media and other substances according to the needs of the invention, e.g. addition of new absorbent into and removal of absorbent laden with carbonaceous chemicals from the photo-biochemical reactor. To maximize efficiency, the absorbent may be recycled within the system after stripping of the carbonaceous chemical therefrom.

As will be appreciated by those of skill in the art, a culture condition sufficient for said photobiocatalyst to convert a feedstock comprising carbon dioxide into said carbonaceous chemical through photosynthesis will vary depending on the nature of the photobiocatalyst, the feedstock, the carbonaceous compound to be synthesized, the properties of the absorbent and the source and flux of the light source for photosynthesis. An appropriate culture condition is readily determined by one of skill.

In an exemplary embodiment that employs cyanobacteria as a photobiocatalyst, culture conditions sufficient for said photobiocatalyst to convert a feedstock comprising carbon dioxide into said carbonaceous chemical through photosynthesis includes BG-11 (ATCC medium 616), a standard medium for “blue-green algae” or other specialized media used in the art and a natural or artificial light source in the 400-700 nm range. In some embodiments, the gas fed to the photo-biochemical reactor comprises about 10-20%, less than about 10%, or less than about 5%, or less than about 1%, by volume of carbon dioxide; the balance of the feed gas may comprise inert gases as further described herein.

The carbonaceous chemical produced in step (a) can undergo one or more post-synthetic modification or processing steps, which can occur in the presence of the absorbent or after separation of the absorbent and the carbonaceous chemical. Exemplary post-synthetic modification steps include, without limitation, reduction, hydrogenation, oxidation, oligomerization or polymerization to form homo- or hetero-oligomers or -polymers, esterification, hydrolysis, amination, carbonylation or decarbonylation. Exemplary post-synthetic processing steps include, without limitation, finishing, distillation, purification, and other conventional processing steps known in the art. Also envisioned herein in various embodiments of the invention is a system including a post-synthetic reactor unit that performs at least one or more of the foregoing modification and processing steps.

Also provided herein is a photosynthetic system for producing, collecting and isolating a carbonaceous chemical from a “photobiocatalyst.” The photobiocatalyst is produced from the culture of a photosynthetic microorganism or component thereof (e.g. Cyanobacteria, an algae or an isolated chloroplast) that has been genetically engineered to maximize carbon flux from photo-synthetically fixed carbon dioxide and uses sunlight to produce a carbonaceous product, e.g. isoprene. Another aspect of the invention is that it contains a mechanism to handle oxygen produced by photosynthesis, in a way that prevents the formation of an explosive mixture.

Yet another aspect of the invention provides a hydrocarbon fuel, e.g. isopentane, derived from the carbonaceous product, e.g. isoprene, of the methods described herein. In still another aspect of the invention, the oil, containing the product hydrocarbon, is sent to a separate system where the volatile hydrocarbon is removed from the oil using heat (“stripping system”). In some embodiments, the stripping system includes equipment such as a column containing appropriate internal elements so as to effect a separation of the volatile hydrocarbon from the oil and from any impurities present thus yielding a substantially pure finished product. Another aspect of the invention would be a subsequent process step wherein hydrogen is added to the product hydrocarbon in the presence of appropriate catalyst(s) so as to yield a finished hydrocarbon product with a lower degree of unsaturation (less double bonds) or to yield a fully saturated product (no double bonds).

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component is labeled in every drawing. In the drawings:

FIG. 1 illustrates an exemplary photo-biochemical reactor comprises an open vessel containing the medium and photobiocatalyst, covered by a layer of oil miscible to isoprene but immiscible to water and oxygen, shielded from the wind and dust by a covering that permits the transmission of light at wavelengths needed for photosynthesis, but blocks or filters out other wavelengths of light that would affect the viability of the photobiocatalyst and/or its ability to produce isoprene, e.g. through excessive heat, UV radiation, etc. Medium containing the photobiocatalyst is the solution of nutrients necessary to sustain the photobiocatalyst in a form optimal for maximal production of the volatile olefin for example isoprene. The photobiocatalyst, grown in a separate photobioreactor, is sent into the open vessel where carbon dioxide, air (as a source of nitrogen), water and nutrients necessary to support the photobiocatalyst are added. A layer of oil flows over the aqueous media containing the photobiocatalyst and travels to a chamber that separates isoprene from the oil. The isoprene is isolated and collected and any non-viable photobiocatalyst is removed. As aqueous media containing the photobiocatalyst is returned to the photobioreactor, carbon dioxide, nutrients, water and oil are added. In some embodiments, the oil being added is recycled back to the photobioreactor after the collection of isoprene therefrom. In other embodiments, a new stream of oil may be used as well.

FIG. 2 shows a cross-sectional view of the photobioreactor portion of the exemplary photo-boichemical reactor shown in FIG. 1.

FIG. 3 illustrates an exemplary photobioreactor comprising a long, convoluted tube made of a material which transmits light at wavelengths needed for photosynthesis, preferably between about 400-700 nm. The tubing contains the photobiocatalyst in aqueous media mixed with oil, which is immiscible with water but miscible with isoprene. Medium containing the photobiocatalyst provides the solution of nutrients necessary to sustain the photobiocatalyst in a form optimal for maximal production of the volatile olefin, for example, isoprene. This media containing photobiocatalyst is pumped through the tubing from a central pumping and separating system where carbon dioxide, water and nutrients necessary to support the photobiocatalyst are added, and oxygen produced as a byproduct of photosynthesis is safely removed in a manner that prevents it from becoming a flammable or explosive hazard. Oil containing isoprene is separated from the aqueous media containing the photobiocatalyst and sent to a means for separating isoprene and oil. As aqueous media containing the photobiocatalyst is returned to the photobioreactor, carbon dioxide, nutrients, water and oil stripped of isoprene are added, and photobiocatalyst which is no longer viable is removed.

FIG. 4 shows an exemplary photobioreactor comprising a plurality of long tubes constructed of polymer film, such as polyethylene, transparent to the wavelengths of light needed for photosynthesis, preferably between about 400-700 nm. The tube is filled with aqueous media and installed at a slight incline to provide the impetus for gases or low density, immiscible liquids, such as isoprene, to travel along the length of the tube to an exit point where the fluids pass into a layer of oil which acts as an absorbent, capturing the isoprene and allowing any gases, including oxygen, to pass through. The media containing the photobiocatalyst receives an inflow of fresh photobiocatalyst, water, and any required nutrients and an exit stream from the photobioreactor removes the net generation of spent photobiocatalyst. Carbon dioxide is added to the photobioreactor and dispersed along the length. (A) provides a side view of the long tube described above. (B) shows the many tubes which make up the photobioreactor are arranged in parallel, producing gases (isoprene and oxygen) that are fed through a central reservoir of oil that captures the isoprene and allows oxygen to bubble through.

FIG. 5 shows an exemplary capture and collection system connected to a pumping and separation system. The capture and collection system contains a vessel in which an aqueous phase containing the photobiocatalyst separates from the isoprene-containing oil phase.

FIG. 6 depicts an exemplary system for isoprene stripping. The system comprises a stripping system which works by removing the more volatile components from liquid by evaporation, a common process applied in the chemical industry. In this system, a liquid comprised of an oil that is significantly less volatile than isoprene, containing a dissolved quantity of isoprene, is fed to a stripping column. In the column, the liquid is distributed over high surface area structures and exposed to heat. The more volatile isoprene leaves the top of the column as a vapor. It enters a heat exchanger where it is condensed and sent to storage. The oil leaves the bottom of the column where a portion is heated and returned to the stripping column. The remaining oil that is substantially free of isoprene is then routed to storage. The isoprene produced is then routed to a hydrogenation unit to produce isopentane.

FIG. 7 provides the nucleic acid sequence of a synthetic isoprene synthase gene (v2.2) and a synthetic isoprene synthase gene.

FIG. 8 provides a simplified reaction scheme for the hydrogenation of isoprene to form isopentane.

FIG. 9 is a flowchart illustrating an exemplary system according to the present invention.

FIG. 10 is a flowchart illustrating an alternative exemplary system according to the present invention.

### DETAILED DESCRIPTION OF THE INVENTION

This invention is not limited in its application to the details of construction and the arrangement of components set forth in the following description or illustrated in the drawings. The invention is capable of other embodiments and of being practiced or of being carried out in various ways. The phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

“Sustainable energy,” as used herein, refers broadly to energy other than fossil fuels. Exemplary sources of sustainable energy include, but are not limited to, solar energy, water power, wind power, geothermal energy, wave energy, and energy produced from other sources, such as wastes and renewables.

As used herein, the term “hydrocarbon compounds” include hydrocarbons and hydrocarbon derivatives, e.g. alcohol, halide, thiol, ether, aldehyde, ketone, carboxylic acid, ester, amine, and amide, etc.

As used herein, the term “carbonaceous chemical,” refers to any carbon-containing chemical that can be produced by a photobiocatalyst. In various embodiments, the carbonaceous chemical is a hydrocarbon, while in other embodiments, the chemical includes one or more heteroatoms, e.g., O, S, N, P and the like. The heteroatoms can be joined to one or more carbon atoms or, when there is more than one heteroatom, they are optionally joined to each other, e.g., SO₃H. The carbonaceous chemical can include residues that are alkyl, heteroalkyl, aryl or heteroaryl residues.

The absorbent of use in exemplary embodiments of the present invention is selected to be essentially miscible with the carbonaceous chemical produced by the bioreactor. As used herein, “essentially miscible” incorporates the standard definition of miscible in which the carbonaceous chemical and the absorbent mix in all proportions to form a homogeneous mixture, and further incorporates absorbent-carbonaceous chemical mixtures in which the absorbent is a solvent for the carbonaceous chemical. With respect to this second aspect of the definition, exemplary absorbents include those able to dissolve up to about 20%, up to about 30%, up to about 40% or up to about 50% of their weight of the carbonaceous chemical. Exemplary absorbents meeting these criteria include, without limitation, organic absorbents and include by way of example, organic solvents, organic oils and organic waxes.

In various embodiments of the invention, the absorbent is “essentially immiscible” with water. As used herein, “essentially immiscible” incorporates the standard definition of immiscible in which water and the absorbent do not mix in any proportions to form a homogeneous mixture, and further incorporates absorbent-water mixtures in which the absorbent is a poor solvent for water. With respect to this second aspect of the definition, exemplary absorbents include those able to dissolve less than about 30%, less than about 20%, less than about 10%, less than about 5% or less than about 1% of their weight of water. Exemplary absorbents meeting these criteria include, without limitation, organic absorbents and include by way of example, organic solvents, organic oils and organic waxes.

In various embodiments, of the invention, the absorbent is “essentially immiscible” with oxygen. As used herein, “essentially immiscible” incorporates the standard definition of immiscible in which oxygen and the absorbent do not mix in any proportions to form a homogeneous mixture, and further incorporates absorbent-oxygen mixtures in which the absorbent is a poor solvent for oxygen. With respect to this second aspect of the definition, exemplary absorbents include those able to dissolve less than about 30%, less than about 20%, less than about 10%, less than about 5% or less than about 1% of their weight of water. Exemplary absorbents meeting these criteria include, without limitation, organic absorbents and include by way of example, organic solvents, organic oils and organic waxes.

In various embodiments of the invention, the absorbent is essentially immiscible with both water and oxygen. In an exemplary embodiment, the absorbent contains no more than from about 0% to about 2% water and no more than from about 0% to about 1% oxygen. In various exemplary embodiments, the absorbent contains from about 0.1% to about 20% of the carbonaceous chemical.

In various embodiments, the method and system of the invention is of use to produce a carbonaceous chemical in an “essentially pure state.” As used herein, the term “essentially pure state,” refers to a purity of at least 80%, at least 85%, at least 90%, at least 95%, at least 96%, at least 97%, at least 98% or at least 99%.

The term “alkyl,” by itself or as part of substituent, means, unless otherwise stated, a straight or branched chain, or cyclic hydrocarbon radical, or combination thereof, which may be fully saturated, mono- or polyunsaturated and can include mono-, di- and multivalent radicals, having the number of carbon atoms designated (i.e. C₁-C₁₀ means one to ten carbons). In some embodiments, the term “alkyl” means a straight or branched chain, or combinations thereof, which may be fully saturated, mono- or polyunsaturated and can include di- and multivalent radicals. Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl” with the difference that the heteroalkyl group, in order to qualify as an alkyl group, is linked to the remainder of the molecule through a carbon atom. Alkyl groups that are limited to hydrocarbon groups are termed “homoalkyl”.

The term “alkenyl” by itself or as part of another substituent is used in its conventional sense, and refers to a radical derived from an alkene, as exemplified, but not limited, by substituted or unsubstituted vinyl and substituted or unsubstituted propenyl. Typically, an alkenyl group will have from 1 to 24 carbon atoms, with those groups having from 1 to 10 carbon atoms being useful examplars.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being useful exemplars in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si, S, B and P and wherein the nitrogen and sulfur atoms may optionally be oxidized and the nitrogen heteroatom may optionally be quaternized. In some embodiments, the term “heteroalkyl,” by itself or in combination with another term, means a stable straight or branched chain, or combinations thereof, consisting of the stated number of carbon atoms and at least one heteroatom. The heteroatom(s) may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —CO₂R′— represents both —C(O)OR′ and —OC(O)R′.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. A “cycloalkyl” or “heterocycloalkyl” substituent may be attached to the remainder of the molecule directly or through a linker, wherein the linker is preferably alkylene. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-tritluoroethyl, 4-chlorobutyl, 3-bromopropyl, and the like.

The term “aryl” means, unless otherwise stated, a polyunsaturated, aromatic, substituent that can be a single ring or multiple rings (preferably from 1 to 3 rings), which are fused together or linked covalently. The term “heteroaryl” refers to aryl groups (or rings) that contain from one to four heteroatoms selected from N, O, S, Si and B, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other tent's (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) are meant to include both substituted and unsubstituted forms of the indicated radical. Exemplary substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: substituted or unsubstituted alkyl, substituted or unsubstituted heteroalkyl, substituted or unsubstituted aryl, substituted or unsubstituted heteroaryl, substituted or unsubstituted cycloalkyl, substituted or unsubstituted heterocycloalkyl, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR′″, —NR—C(NR′R″)=NR′″, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the present inventions includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: substituted or unsubstituted alkyl, substituted or unsubstituted heteroalkyl, substituted or unsubstituted aryl, substituted or unsubstituted heteroaryl, substituted or unsubstituted cycloalkyl, substituted or unsubstituted heterocycloalkyl, —OR′, ═O, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR′″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the present inventions includes more than one R group, for example, each of the R groups is independently selected as are each R′, R″, R′″ and R″″ groups when more than one of these groups is present.

Two of the substituents on adjacent atoms of the aryl or heteroaryl ring may optionally be replaced with a substituent of the formula -T-C(O)—(CRR′)_{q}-U-, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “acyl” describes a substituent containing a carbonyl residue, C(O)R. Exemplary species for R include H, halogen, substituted or unsubstituted alkyl, substituted or unsubstituted aryl, substituted or unsubstituted heteroaryl, and substituted or unsubstituted heterocycloalkyl.

As used herein, the term “fused ring system” means at least two rings, wherein each ring has at least 2 atoms in common with another ring. “Fused ring systems” may include aromatic as well as non aromatic rings. Examples of “fused ring systems” are naphthalenes, indoles, quinolines, chromenes and the like.

As used herein, the term “heteroatom” includes oxygen (O), nitrogen (N), sulfur (S), silicon (Si) and boron (B).

The symbol “R” is a general abbreviation that represents a substituent group. Exemplary substituent groups include substituted or unsubstituted alkyl, substituted or unsubstituted heteroalkyl, substituted or unsubstituted aryl, substituted or unsubstituted heteroaryl, and substituted or unsubstituted heterocycloalkyl groups.

### Overview

Embodiments of the present invention provide new energy solutions that are sustainable both environmentally and economically. The present invention provides methods and systems for producing, collecting and isolating valuable products from the use of a “photobiocatalyst.” In exemplary embodiments, the present invention provides methods and systems for preparing a carbonaceous chemical. The method includes maintaining a photobiocatalyst under a culture condition sufficient for said photobiocatalyst to convert a feedstock comprising carbon dioxide into said carbonaceous chemical through photosynthesis. During at least a portion of the period during which the carbonaceous material is being foiined, the photobiocatalyst is maintained in a culture medium in contact with an absorbent medium essentially miscible with said carbonaceous chemical.

In some embodiments of the invention, the system includes a photo-biochemical reactor and optionally a photo-bioreactor. The system may include one or a plurality of these components, as well as additional components, arranged in series or in parallel. The “photo-biochemical reactor” exposes the photobiocatalyst to light, a source of carbon dioxide, and H₂O to drive the photosynthetic process in the photobiocatalyst and thereby convert carbon dioxide into a carbonaceous chemical that is collected and isolated and optionally directed to further processing. A high level block diagram of exemplary embodiments of the invention is provided in FIG. 9 and more specific illustrations of exemplary embodiments of processes and apparatus are presented in subsequent figures and described in detail below. In other embodiments of the invention, the photobiocatalysts are grown in a “photo-bioreactor” and then transferred to a “photo-biochemical reactor” for the production and isolation of a product hydrocarbon, e.g. a volatile olefin, e.g. isoprene, and incubated under conditions where it does not grow, or grows very slowly, when used as a catalyst.

As used herein, the team “photobiocatalyst” refers to a genetically engineered photosynthetic microorganism or chloroplast that is capable of producing useful hydrocarbons, such as isoprene, from carbon dioxide (CO₂). Preferably, the “photobiocatalyst” does not grow, or grows very slowly, when used as a catalyst, but is manufactured separately by the culture of a genetically engineered photosynthetic microorganism, algae or algae containing genetically engineered chloroplasts or isolated genetically engineered chloroplasts. In other embodiments, the photobioreactor is optimized for maximal growth of photobiocatalyst and generally does not contain a means for capturing and isolating isoprene. In still other embodiments, the photobioreactor used to grow the photobiocatalyst is the same apparatus as the photo-biochemical reactor and also contains a means for capturing and isolating isoprene.

A wide variety of water-immiscible carbonaceous chemicals, e.g. hydrocarbon compounds, can be produced by genetically engineered cyanobacteria, which pass readily out of the cyanobacteria. These hydrocarbons contain more energy per unit mass than either alcohols or triglycerides. Exemplary products derivable from the inventive processes include, without limitation, isoprene, and other useful intermediates optionally removed at various stages, which can be used directly or further processed into usable forms of energy, i.e. as a feed or as a fuel. For example, isoprene contains approximately one hundred and fifty percent (150%) of the potential combustion energy per kg of ethanol. The direct release of water-immiscible hydrocarbons into surrounding fluid eliminates the need and consequent cost of separation from the cyanobacteria and aqueous media that is required to isolate triglycerides produced in either cyanobacteria or algae. A favourable consequence of this is that this direct release enables the genetically modified cyanobacteria to function as catalysts that are not consumed in the process, and remain active even when in a stationary phase of growth, minimizing the consumption of energy for the generation of non-product cellular components. Such water immiscible hydrocarbons can be separated from the oxygen co-product of photosynthesis using low cost absorption.

The photo-biochemical reactor generally is comprised of mechanical components to fulfil one or more of the following functions: (1) to provide CO₂ (with or without inert gases such as nitrogen), water and nutrients to the cyanobacteria to maintain their viability and optimize production of the desired carbonaceous product or volatile and/or immiscible carbonaceous chemical; (2) to allow the photobiocatalysts to consume large quantities of CO₂ from the atmosphere or another CO₂ rich source; (3) to incubate the photobiocatalyst under conditions that allow the capture of isoprene or other volatile and/or immiscible carbonaceous chemical produced; (4) to prevent explosive mixtures of the volatile isoprene or other volatile and/or immiscible carbonaceous chemical and oxygen formed through photosynthesis from developing in a confined space; (5) to prevent over-heating of the culture of photobiocatalysts due to the direct exposure to sunlight; and (6) to facilitate the isolation and purification of isoprene or other volatile and/or immiscible olefin in large quantities; (7) to hydrogenate isoprene or other hydrocarbon compound or carbonaceous chemical produced by the present invention.

The photo-biochemical reactor preferably is constructed of materials and in such a manner as to be justified by the quantities of volatile immiscible hydrocarbon and is present in a part of the world that provides the right combination of temperature and total sunlight to operate at a useful efficiency. The photo-biochemical reactor should have sufficient strength to withstand pressure generated by the gas phase when the reactor is in an operating state.

In exemplary embodiments of the invention, the system comprises one or more “product absorbers” arranged in series or in parallel. The product absorber can be an integrated component of the photo-biochemical reactor(s) or maintained as a distinct structure within the system. In some embodiments, the product absorber utilizes a water-immiscible solvent to separate the volatile hydrocarbon produced by the photobiocatalyst from the oxygen concomitantly produced and/or capture the product hydrocarbon. Examples of solvents suitable for use as a product absorbent include, without limitation, branched alkanes, e.g. isoparaffinic hydrocarbons, e.g. ExxonMobil's Isopar L™, and terpenes, e.g. monoterpenes, e.g. limonene. One or more product absorbers can be arranged in series or in parallel with each individual photo-biochemical reactor or group of photo-biochemical reactors.

In an exemplary embodiment of the invention, the system comprises a product absorber which comprises a system for the intimate contact of liquid organic absorbent with the incoming vapor stream to effect a near complete recovery of the product, e.g. isoprene contained therein, thereby yielding an exit gas that is essentially free of isoprene. Preferably, liquid organic absorbent is fed at the upper region of the absorber. In some embodiments, the product absorber is filled with packing for increased surface area to effect extractive distillation of the isoprene. Suitable packing materials for use in the present invention would be known to those of ordinary skill in the art. The outgoing liquid organic absorbent with trapped isoprene from the product absorber can be stored, subjected to the product recovery process described herein, and/or recycled within the system of the present invention. In some embodiments, vapor exiting the product absorber is directed to further isoprene recovery or fed to a combustion process. The oxygen by-product released by the photo-biocatalyst may be vented. In some embodiments of the invention, the product absorber does include a heat exchanger and/or internal structures to facilitate mass transfer.

Absorbent substances of the same or different composition can be used for the various absorbers within a system in configurations providing more than one absorber. For example, a liquid organic absorbent containing low levels of isoprene can be fed to an initial product absorber continuously or intermittently whereas a second liquid organic absorbent containing little or no isoprene is fed to a secondary product absorber. In other embodiments, a liquid organic absorbent that is essentially free of isoprene is used in both the initial and secondary product absorber.

Except as otherwise noted, the apparatus and system of the invention can be assembled from conventional processing equipment that is readily and commercially available. The equipment, reactors, ancillary systems, and process lines can be constructed using, where applicable, any gas-impermeable and water-resistant material known in the art. In exemplary embodiments, the apparatus of the invention is constructed primarily of carbon steel. While more exotic metals can be used, they are not absolutely necessary to achieve the objects and advantages of the invention. Examples of exotic metals that can be used include Hastelloy, tantulum, and various hardened steels for acid service, for control valve trim and for grinding equipment. Those of ordinary skill in the art will recognize that unnecessary piping runs and individual process components can be eliminated to allow for even longer uninterrupted processing runs and greater efficiency.

### Photosynthetic Microorganisms Genetically Engineered to Produce Isoprene by Introducing the Plant Biosynthetic Enzyme Isoprene Synthase

In one aspect, the present invention provides a photocatalyst comprising photosynthetic microorganisms genetically engineered to produce isoprene by introducing a plant biosynthetic enzyme, e.g. isoprene synthase.

In some embodiments, the genetically engineered photosynthetic microorganism produces hemiterpenes (for example isoprene) and terpenes from precursors generated via the 2-C-methyl-D-erythritol-4-phosphate (MEP) metabolic pathway and/or the mevalonate pathway. A wide variety of cyanobacteria can be genetically modified, including but are not limited to, thermophilic cyanobacteria, such as Thermosynechococcus (T.) elongatus BP-1, and cyanobacteria of the genera Synechococcus, Synechocystis and Anabaena, including the species Synechocystis Sp. PCC 6803 and Anabaena 7120. The cyanobacteria are genetically engineered through the introduction of a gene encoding a terpene synthase, for example isoprene synthase. The encoded isoprene synthase can be from any source, provided that it is functional (exhibits activity) in the genetically engineered microorganism (e.g., cyanobacterium) into which it is introduced. Suitable sources of isoprene synthase include, but are not limited to, plants, bacteria, fungi, and other non-mammalian sources and mammalian sources. In a particular embodiment, the isoprene synthase gene is a plant isoprene synthase gene, such as a tree isoprene synthase gene. In one embodiment, the isoprene synthase gene is a poplar (Populus albaxPopulus tremula) isoprene synthase gene. The isoprene synthase gene can be modified, before or after introduction into cyanobacteria, for optimization of expression and/or enzymatic activity in cyanobacteria. In one embodiment, the gene encoding isoprene synthase is integrated into the cyanobacterial genome, while in another embodiment, the gene encoding isoprene synthase is carried on a plasmid contained in the genetically engineered photosynthetic microorganism (e.g. cyanobacterium).

Genetically modified cyanobacteria, and the methods of producing isoprene and using such genetically modified cyanobacteria are described in WO2008/137092 A2, which is incorporated herein by reference in its entirety.

In an alternative embodiment, the photosynthetic microorganism is one which, in the absence of modification, produces substantially no isoprene and is genetically engineered so that a normally silent endogenous isoprene synthase gene is activated and/or constitutively expressed and functions to produce isoprene.

Controlling the Division of Genetically Modified Isoprene Producing Cyanobacteria or Algae to Optimize their Production of Isoprene in Closed Systems

In another aspect, the present invention provides genetic modifications of the photobiocatalyst to maintain constant cell density and cell size such that most energy is expended in biosynthesis of desired carbon-containing product in response to a controllable signal. The controllable signal, alone or in combination with changes in environment or culture conditions of the photobiocatalyst, enables the photobiocatalyst to maintain constant cell density and cell size such that most energy is expended in biosynthesis of desired carbon-containing product.

It is generally difficult to stop a cell from growing and dividing without dying, as it will eventually experience oxygen damage. The cell, therefore, needs to renew itself periodically. To circumvent this problem, the present invention provides methods to slow renewal whereby the biocatalyst is maintained in a weak chemostat by utilizing a small liquid flow that removes cells at a rate that enables cell renewal, but maintains them at a certain density.

In one aspect, the present invention provides methods to limit the cell growth and/or cell division of photobiocatalysts. The methods include, but are not limited to, physically induced methods such as depriving cells of key nutrients required for generation of biomass, changing the pH range to disfavour generation of biomass, and changing the temperature.

Other aspects of this invention relate to additional genetic modifications of the photobiocatalyst, beyond those necessary for isoprene production, that reduce or eliminate expression of genes required for the generation of biomass or for cell division. In some embodiments, the genetic modifications are the introduction of genes under the control of inducible or constitutive promoters or regulatory sequences. In some embodiments, the cell growth and/or division are inhibited when the optimal cell size/and or density has been reached. Aspects of the invention relate to the use of inducible promoters to regulate expression of key hydrocarbon forming genes. Inducible promoters include, but are not limited to, pNir and pPetE promoters in cyanobacteria, which are activated by addition of nitrate and copper ions, respectively. These promoters can, once activated, complete the genetic machinery required for the activation of the desired metabolic pathway, shunting ATP from biomass growth to biosynthesis of the desired carbon containing product. In one embodiment, pPetE is used to drive isoprene synthase expression, which upon activation by copper ions completes the Methyl Eryithritol Pathway (MEP) that requires at least five ATP to produce isoprene. The level of activity of the pathway is further regulated by titration with copper ions, with higher concentration driving higher expression levels.

In another aspect, the present invention provides methods to inhibit cell division that involving inhibition or limiting expression of genes required for cell division. The circadian clock in cyanobacteria is independent of cell division, thus it is possible to inhibit cell division without affecting ingrained circadian rhythm functions (Johnson (2007) Cold Spring Farb Symp Quant Biol 72:395-4040). However, in most instances when cell division is inhibited, cell size increases. Therefore, a careful balance between cell growth and cell division need to be struck, with overgrowing cells being removed constantly. In one embodiment, the gene FtsZ, is manipulated (Saks et al., (2006) J. Bacteriol. 188:5958-5965). Generally, the inhibition of FtsZ, is carefully titrated.

### Selection of the Optimal Species of Cyanobacteria to Have All of the Properties Necessary to Function Optimally as Photobiocatalysts in a Photo-biochemical Reactor

In another aspect, the present invention provides the selection of slowly dividing photobiocatalyst during the production of isoprene. This is desirable because once an optimal density for volatile immiscible olefin production is reached, the culture is more easily maintained at that optimal density. This is particularly important during continuous-type production. Preferably, a photobiocatalyst that grows rapidly, either in a separate photobioreactor or in the photo-biochemical reactor, is used during a replenishment phase and then the same species is induced to grow much slower during a production phase. Species of cyanobacteria naturally vary in the time to divide. On average, a cyanobacterium divides approximately once every 10 hours. Species that take longer than average are generally favored over those that inherently divide rapidly during the production phase.

In another aspect, the present invention provides methods for genetically modification of the photosynthetic microorganism of choice to have the ability to produce or produce more isoprene. In some embodiments, cyanobacteria Thermosynechococcus elongatus or species in the genera Synechocystis, Synechococcus and Anabaena are genetic modified to optimize the production of isoprene.

In some embodiments, the cyanobacteria are capable of growing in extremely simple media. Cyanobacteria can be divided into those that are nitrogen fixing and others that are non-nitrogen fixing. For example, Thermosynechococcus elongatus, Synechococcus and Synechocystis Sp. PCC 6803 cannot fix nitrogen. Cyanobacteria in the genera Chlorogleopsis, Fischerella and Anabaena are nitrogen fixers and do not require an additional nitrogen source in the media other than nitrogen from the air. The advantage of using a species that is a nitrogen fixer is to avoid the expense of providing nitrate to the photo-biochemical reactor. Apart from this distinguishing feature, most other cyanobacteria grow in similar media.

In some embodiments, the cyanobacteria are capable of growing in salt water. Salt water is more abundant than fresh water, and is a less precious resource for the sustenance of terrestrial life. Various species of cyanobacteria differ greatly in their tolerance for salt. For example, Synechococcus elongatus PCC 6301, Synechococcus elongatus PCC 7942, Synechocystis sp. PCC 6803, Thermosynechococcus elongatus BP-1, Anabaena sp. L-31 and R-cyanobacteriam Aphanocapsa all generally prefer freshwater. Anabaena torulosa is a salt-tolerant brackish water strain, whereas other species such as Synechococcus WH8012 flourish in salt water.

In some embodiments, the cyanobacteria tolerate relatively high temperatures. The photobiocatalyst of the invention generally is exposed to direct sunlight, under glass or plastic all day every day, which is likely to heat the media containing the cyanobacteria by a classic “green house effect”. There is a number of advantages to situating a photo-biochemical reactor in parts of the world that are close to the equator, which get large amounts of sunlight, and are consequently very warm. One way of dealing with the relatively high daytime temperatures is to fill the photobioreactors with a sufficiently large volume of media to buffer extreme changes in temperature. Another way that may be used in conjunction with the first is to use species of cyanobacteria known as thermophiles, organisms that live and thrive at relatively high temperatures. Preferably, the photobiocatalyst of the invention can tolerate the highest temperature in the photobioreactor without the need for expensive cooling. Thermophilic cyanobacteria have a particularly high heat tolerance. Suitable thermophilic cyanobacteria include, but are not limited to: Thermosynechococcus elongatus BP-1, Chlorogleopsis species and Fischerella species. If a photo-biochemical reactor is situated in a more temperate part of the world a mesophilic cyanobacteria could be used. A mesophile is an organism, especially a microorganism that lives and thrives at moderate temperatures. Cyanobacterial examples of mesophiles include species belong to the genera Synechococcus and Anabaena.

In some embodiments, the present invention provides cyanobacteria that are extremophile. The extremophile is identified and characterized using technology known in the art and is used as the basis for building photobiocatalysts. Extremophiles thrive under conditions of extremely high salt concentration or temperature. The fact that they have evolved to survive under such extreme conditions make them ideally suited to be used in industrial processes such as a photo-biochemical reactor. These extremophiles are sequenced and annotated, and biochemically characterized before being exploited as hosts for isoprene synthase.

In some embodiment, the cyanobacteria of the invention preferably survive above the boiling point of isoprene which is 34° C. These cyanobacteria include, but are not limited to: Thermosynechococcus elongatus, Chlorogleopsis and Fischerella, all of which are thermophiles with growth optima above 34° C. In some embodiments, Synechocystis is used, which grows up to 45° C. In some embodiments, Anabaena is used, which grows best below 30° C. but is capable of growth above 34° C.

### Utilizing Isolated Chloroplasts as the Photobiocatalyst

In another aspect, the present invention provides a photobiocatalyst comprising isolated chloroplasts. When stably integrated into the chloroplast genome, transgenes express large amounts of foreign proteins (De Cosa et al (2001), Nat. Biotechnol. v19, p 71-74). This is due to the presence of up to 10,000 copies of the chloroplast genome in each plant cell (Grevich & Daniell (2005), Crit. Rev. Plant Sci., v24, p 238-245; Daniell et al (2005), Trends Biotechnol., v23, p 238-245). In some embodiments, transgenes are integrated at a precise location in the genome by homologous recombination, which is mediated by flanking chloroplast DNA sequences present in the chloroplast vector. This generally eliminates position effects frequently observed in nuclear transgenic lines (Daniell et al (2005), Trends Biotechnol., v23, p 238-245). Targeting sequences are generally 1 kb in size and are located on either side of the expression cassette, which is inserted using a suitable restriction enzyme in the spacer region of the targeting sequence. Transgenes may be integrated into three types of spacer regions. Transcriptionally silent spacer regions are found at sites where chloroplast genes are located on opposite DNA strands. Read-through spacer regions are found between chloroplast genes located on the same strand and where each gene has its own promoter. Transcriptionally active spacer regions are found in chloroplast operons in which a single promoter drives transcription of several genes. In some embodiments, the region used for integration is the transcriptionally active spacer region between the trnI and trnA genes. This region is located within the rRNA operon, where the 16S rRNA promoter drives transcription of six genes and each spacer region within this operon is transcriptionally active. The trnl gene intron also contains a chloroplast origin of replication, which facilitates replication of foreign vectors within chloroplasts and enhance the probability of transgene integration (Daniell et al (1990), Proc. Natl. Acad. Sci. USA, v87, p 88-92; Kunnimalaiyaan et al (1997), Nucleic Acids Res., v25 p 2681-3686). Transcriptionally active spacer regions also offer the unique advantage that transgenes lacking promoters or 5′- or 3′-untranslated regions (UTRs) can be inserted and expressed. However, other spacer regions (transcriptionally silent or read-through) may also be used.

Other advantages of transplastomic over nuclear transgenic include lack of transgene silencing. For example, in plant, there is no transgene silencing despite 169-fold higher levels of transgene transcript than in nuclear transgenic plants (Dhingra et al (2004), Proc. Natl. Acad. Sci. USA, v101, 6315-6320), and foreign protein levels up to 46% (wt/wt) of total leaf protein (De Cosa et al (2001), Nat. Biotechnol. v19, p 71-74). For another example, multivalent vaccines can be engineered in a single transformation step because polycistrons are translated without processing into monocistrons; several heterologous operons have been expressed in transgenic chloroplasts (Quesada-Vargas et al (2005), Plant Physiol., v138, p 1746-1762).

In some embodiments, the DNA is delivered using gene gun or biolistic technology. Particle bombardment, in which small gold or tungsten particles are coated with DNA and shot into young plant cells (leaf or callus tissue), is widely used and effective method for transforming plastids. The first successful chloroplast transformation using this method was reported in Chlamydomonas, an algae that is a potential Photobiocatalyst, by complementation of a native gene fragment in a deletion mutant (Boynton et al, (1988), Science, v240, p 1534-1538).

In some embodiment, the DNA is delivered using PEG-mediated transformation (Golds et al, (1993), Nat. Biotechnol., v11, p 95-97). After protoplast isolation, PEG incubation with foreign DNA facilitates entry of DNA through cell and chloroplast membranes.

Other suitable methods for of chloroplast transformation technology, including a detailed protocol for the construction of chloroplast expression and integration vectors, selection and regeneration of transfoimants, evaluation of transgene integration and inheritance, confirmation of transgene expression and extraction, and quantitation and purification of foreign proteins, are disclosed in Verma et al, (2008), Nat. Protocols, v3 p 739-758, which is incorporated by reference in its entirety.

In some embodiment, photosynthesis is measured in isolated intact chloroplasts trapped in the cavities of membrane filters. Thin layers of chloroplasts so obtained are assayed for oxygen production and carbon dioxide assimilation in leaf chambers. Photosynthetic gas exchange was demonstrated to take place. The chloroplasts were morphologically intact as shown by light and scanning electron microscopy and displayed stable rates of photosynthesis (Cerovic et al (1987), Plant Physiol., v84, p 1249-1251).

In one embodiment, the photobiocatalyst comprises a genetically engineered chloroplast to contain all of the genes necessary to produce isoprene from carbon dioxide including the gene encoding isoprene synthase. The chloroplast is replicated in a photosynthetic eukaryote, for example Chlamydomonas, but isolated and maintained in isolation, such as on a membrane, for the production of isoprene from carbon dioxide using energy from sunlight.

### Optimal Culture Conditions for the Production of the Photobiocatalyst

In another aspect, the present invention provides optimal culture conditions for the production of the photobiocatalyst. All characterized species of cyanobacteria have been successfully cultured on at least the laboratory scale. Many of these grow on standard medium for “blue green algae” such as BG-11 (ATCC medium 616). Also, many specialized culture media have been developed. Cyanobacteria of the present invention can be cultured in standard or specialized media, including but are not limited to: Aiba and Ogawa (AO) Medium, Allen and Anion Medium plus Nitrate: ATCC Medium 1142, Antia's (ANT) Medium, Aquil Medium, Ashbey's Nitrogen-free Agar, ASN-III Medium, ASP 2 Medium, ASW Medium: Artificial Seawater and derivatives, ATCC Medium 617: BG-11 for Marine Blue-Green Algae; Modified ATCC Medium 616 [BG-11 medium], ATCC Medium 819: Blue-green Nitrogen-fixing Medium; ATCC Medium 616 [BG-11 medium] without NO3, ATCC Medium 854: ATCC Medium 616 [BG-11 medium] with Vitamin B12, ATCC Medium 1047: ATCC Medium 957 [MN marine medium] with Vitamin B12, ATCC Medium 1077: Nitrogen-fixing marine medium; ATCC Medium 957 [MN marine medium] without NO3, ATCC Medium 1234: BG-11 Uracil medium; ATCC Medium 616 [BG-11 medium] with uracil, Beggiatoa Medium: ATCC Medium 138, Beggiatoa Medium 2: ATCC Medium 1193, BG-11 Medium for Blue Green Algae: ATCC Medium 616, Blue-Green (BG) Medium, Bold's Basal (BB) Medium, Castenholtz D Medium, Castenholtz D Medium Modified: Halophilic cyanobacteria, Castenholtz DG Medium, Castenholtz DGN Medium, Castenholtz ND Medium, Chloroflexus Broth, Chloroflexus Medium: ATCC Medium 920, Chu's #10 Medium: ATCC Medium 341, Chu's #10 Medium Modified, Chu's #11 Medium Modified, DCM Medium, DYIV Medium, E27 Medium, E31 Medium and Derivatives, f/2 Medium, f/2 Medium Derivatives, Fraquil Medium: Freshwater Trace Metal-Buffered Medium, Gorham's Medium for Algae: ATCC Medium 625, h/2 Medium, Jaworski's (JM) Medium, K Medium, L1 Medium and Derivatives, MN Marine Medium: ATCC Medium 957, Plymouth Erdschreiber (PE) Medium, Prochlorococcus PC Medium, Proteose Peptone (PP) Medium, Prov Medium, Prov Medium Derivatives, S77 plus Vitamins Medium, S88 plus Vitamins Medium, Saltwater Nutrient Agar (SNA) Medium and Derivatives, SES Medium, SN Medium, Modified SN Medium, SNAX Medium, Soil/Water Biphasic (S/W) Medium and Derivatives, SOT Medium for Spirulina: ATCC Medium 1679, Spirulina (SP) Medium, van Rijn and Cohen (RC) Medium, Walsby's Medium, Yopp Medium, Z8 Medium (http://www-cyanosite.bio.purdue.edu/media/table/media.html and (Castenholz, R. W., Methods Enzymol. 1998; 167: 68-93)). Culturing of cyanobacteria is conducted according to standard methods, which are known to those of skill in the art (Rogers, L. J., and Gallon, J. R., “Biochemistry of the Algae and Cyanobacteria”, Clarendon Press, Oxford, 1988; Burlew, J. S., “Algal Culture: From Laboratory to Pilot Plant”, Carnegie Inst. Washington, Publication 600, Washington D.C., 1961; Round, F. E., “Biology of Algae”, St. Martin's Press, New York, 1965). These conditions include sufficient light, since the microorganisms are photosynthetic microorganisms.

The media used in the photo-biochemical reactor generally is modified depending on whether or not the species of cyanobacteria can fix inorganic nitrogen or not.

Optimal Culture Conditions for the Culture of the Photobiocatalyst during the Production of Isoprene

The present invention provides optimal culture conditions for the culture of the photobiocatalyst during the production of a carbonaceous chemical, e.g. hydrocarbon compound, e.g. isoprene. In embodiments yielding isoprene, the very low solubility of isoprene in water suggests a low cell toxicity. Isoprene passes directly through the cell membranes of the photobiocatalyst into the aqueous medium and then gets captured in an isoprene-miscible oil, e.g. Isopar L™.

The media used in the photo-biochemical reactor may be modified depending on the promoters used to drive expression of the genes introduced into the photobiocatalysts to produce the desired hydrocarbon product, e.g. isoprene, and optimize the production thereof. While the process of the invention can be performed across a range of parameters depending on the type of photobiocatalyst employed, certain refinements of the operating conditions such as temperature, pH, and pressure, can be made to enhance the yield and efficiency of the process. It is to be understood that the operating parameters in the present invention may be adjusted in one or more instances in order to accommodate different types of photobiocatalyst. In some embodiments, the gas fed to the photo-biochemical reactor comprises about 10-20%, less than about 10%, less than about 5%, or less than about 1%, by volume of carbon dioxide; the balance of the feed gas may comprise inert gases to dampen the flammability of oxygen and enable agitation of the media in the photobioreactor without introducing so much carbon dioxide that the pH is reduced to levels inhospitable to the photobiocatalyst. As used herein, an “inert gas” is any gas, elemental or molecular, that is not significantly reactive under normal circumstances (e.g., noble gases, gaseous nitrogen). In other embodiments, the feed gas further comprises water vapor, nitrogen, and argon. In still other embodiments, the feed gas comprises essentially pure carbon dioxide.

In embodiments where inert gases are fed to the system with carbon dioxide, the quantity and composition of inert gases can be varied to utilize the most economical source of carbon dioxide and/or to provide sufficient gas flow to optimize the production of the desired hydrocarbon product, e.g. isoprene, agitate the media, prevent evolution of an explosive gas mixture and/or control the media's pH. In one embodiment, genes involved in the production of isoprene are under the control of an inducible promoter, such as pNlr, which drives expression of the gene encoding nitrate reductase. This promoter is not active in cells growing on ammonia as nitrogen source but becomes strongly active in cells growing on nitrate. This is preferred for short-tens batch process, but generally is not preferred for long term because the nitrate is consumed during growth. In another embodiment, promoter pPetE, which drives expression of the gene encoding plastocyanine, is used to drive genes necessary for the production of isoprene. The pPetE promoter is induced by low levels of copper ions (Cu²⁺) in the growth medium. An advantage of this system is that induction is continuous, since Cu²⁺ is not consumed. In addition, the induction level can be titrated by increasing the level of Cu²⁺ in the sub-micromolar range.

In some embodiments, the photobiocatalyst is maintained in stationary phase culture media and conditions. Exemplary media useful for this purpose include various medium lacking one or more key nutrients, e.g. BG-11 medium without nitrate or phosphate. Where oil is used in the method and system of the invention, it is neither used as a source of nutrients for the photobiocatalyst, a manner of effecting the amount of acetyl-CoA in the host cells, nor as a carbon-source for the production of the carbonaceous chemical described herein.

### Optimal Photobioreactor to Generate the Photobiocatalyst

In one aspect, the system of the present invention comprises a bioreactor, such as a photobioreactor. By “photobioreactor” herein is meant an apparatus containing, or configured and dimensioned to contain, a liquid medium for growing phototrophic organism. The photobioreactor has a source of light capable of driving photosynthesis associated therewith, or has at least one surface at least a portion of which is made of a translucent or transparent material. In some embodiments, the material is transparent to light of a wavelength capable of driving photosynthesis (i.e., light of a wavelength between about 400-700 nm, which is emitted by the sun or another light source). Photobioreactor parameters that can be optimized, automated and regulated for production of photosynthetic organisms are further described in (Pulz (2001) Appl Microbiol Biotechnol 57:287-293), the teachings of which are hereby incorporated by reference in their entirety. Such parameters include, but are not limited to, materials of construction, efficient light incidence into reactor lumen, light path, layer thickness, oxygen released and ratio of CO₂/O₂, salinity and nutrients, pH, temperature, turbulence and optical density.

The photobioreactor structure of the present invention can be of various geometries, including, but not limited to, plastic film tubes positioned at an angle, tubular, cylindrical, floating lattices, vertical columns, helical, and flat or tilted grid structures. Examples of commercial development of photobioreactors (Olaizola (2003) Biomolecular Engineering, 20:459-466) and commercial applications of the propagation of prokaryotic and eukaryotic photosynthetic organisms (Spolaore, et al., (2006) Plant physiology and biochemistry 101:87-96) have been described in the literature. Examples of photobioreactor applications for exploiting cyanobacteria in photobioreactors to produce extracellular metabolites are production of ethanol (US Application 20080153080) and hydrogen production (Sakurai and Masukawa (2007) Marine Biotechnology 9: 128-145)).

The photobioreactor of the invention also has inlet and outlet ports providing access, and, optionally, means for mixing an algae media disposed inside the photoreactor. The means for mixing can be a mechanical mixer or air bubbles introduced into the reactor or adopt the form of any suitable structure for supporting mixing as known to those of ordinary skill in the art.

In some embodiments, a first photobioreactor is used to grow the photobiocatalyst, separately from the photo-biochemical reactor. The first photobioreactor is optimized for maximal growth of photobiocatalyst and generally does not contain a means for capturing and isolating isoprene. In other embodiments, the photobioreactor used to grow the photobiocatalyst is the same apparatus as the photo-biochemical reactor and also contains a means for capturing and isolating isoprene.

### Optimal Photobioreactor to Form Part of the Photo-biochemical Reactor Designed to Produce and Capture Isoprene

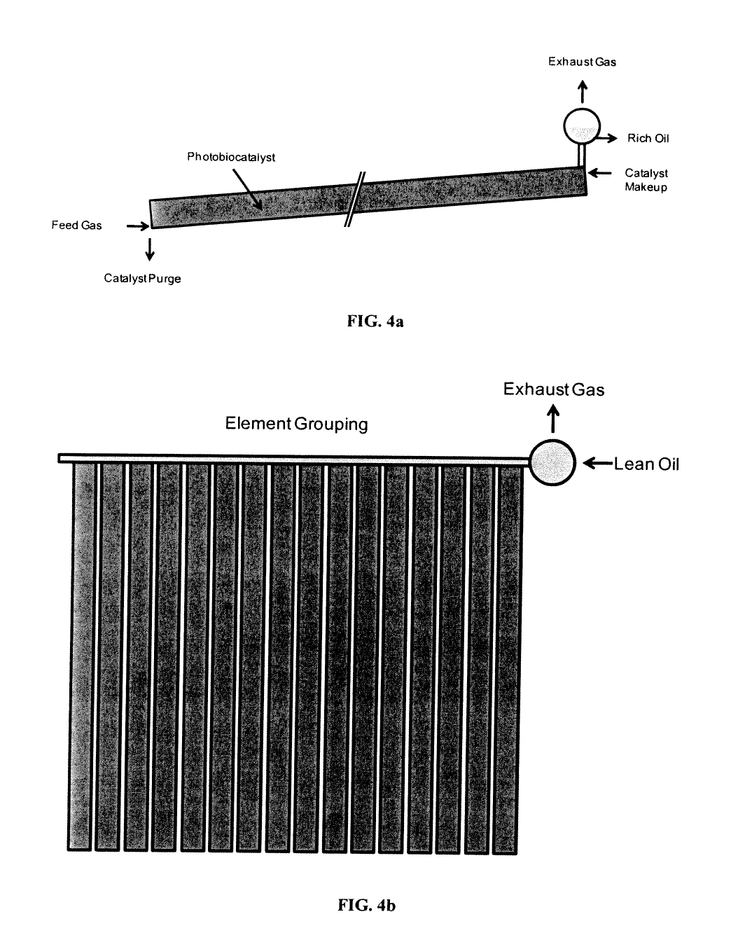

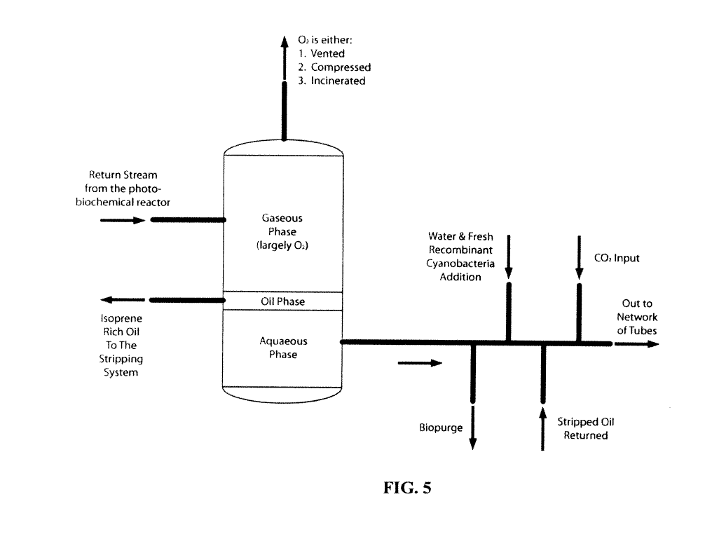

In some embodiments, the system of the present comprises a second bioreactor, such as a photobioreactor, to culture the photobiocatalyst under conditions that maintain viability with a means for capturing, isolating and collecting isoprene. Examples of photobioreactors are described above. In one embodiment, the photobioreactor comprises a plurality of long tubes constructed of polymer film such as polyethylene, transparent to the wavelengths of light needed for photosynthesis, preferably between about 400-700 nm. The tube is filled with aqueous media and installed at a slight incline which provides the impetus for gases or low density, immiscible, liquids, such as isoprene to travel along the length of the tube to an exit point where the fluids pass into a layer of oil which acts as an absorbent, capturing the isoprene and allowing any gases, including oxygen to pass through. The media containing the photobiocatalyst receives an inflow containing fresh photobiocatalyst, water, and any required nutrients and an exit stream from the photobioreactor removes the net generation of spent photobiocatalyst. Carbon dioxide is added to the photobioreactor and dispersed along the length (FIG. 4A and FIG. 4B).

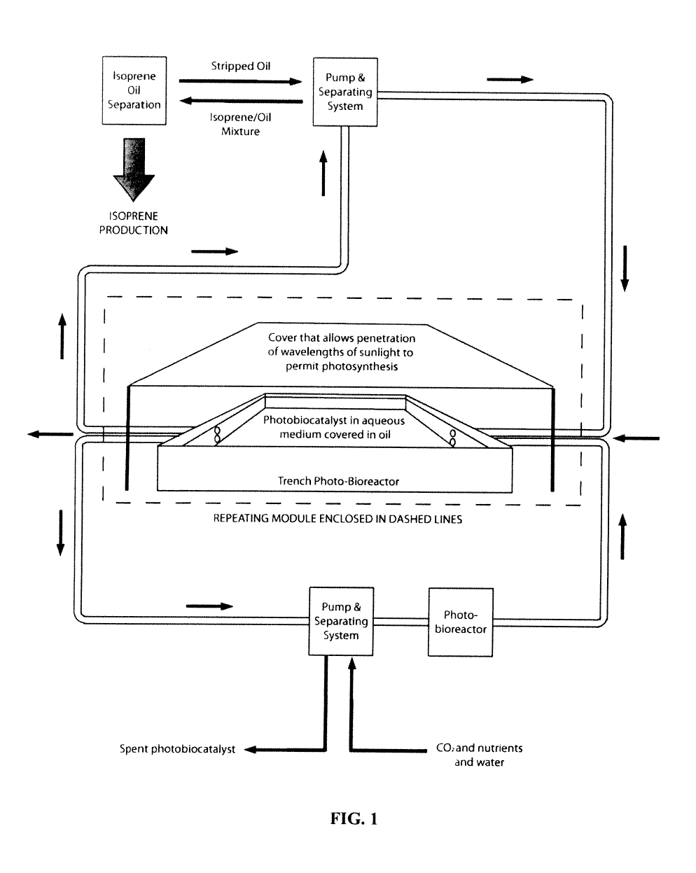

In another embodiment, the photo-biochemical reactor comprises an open vessel. The term “open vessel,” as used herein, refers to a structure that can hold a liquid and has least one opening. The open vessel can be a structure placed above ground (e.g. an above ground tank), partly above the ground (a half buried tank), at the ground level (e.g. a trench) or below the ground. In some embodiments, the open vessel takes the form of a trench, with or without a cover. The open vessel contains water, media and photobiocatalyst covered by an oil which allows O₂ through, but absorbs isoprene produced. The open vessel comprises a cover, which is constructed of, in some embodiments, cost-effective materials that can be further optimized for transmission of light at wavelengths necessary for photosynthesis and filtering out other wavelengths of sunlight which heat the photobioreactor. See FIG. 1 for an exemplary embodiment having this functionality.

In one embodiment, the oil which is immiscible with water but miscible with isoprene is an isoparaffinic fluid, for example Isopar L™, the heaviest in a range of isoparaffinic fluids produced by Exxon Mobile Fluids with a flashpoint of 129° C. (www.isopar.com/Public_Products/Fluids/Aliphatics/Worldwide/Grades_and_Datasheets/Fluids_Aliphatics_Isopar_Grades_WW.asp). In other embodiments, other isoparaffinic fluids in the Isopar range are used. In other embodiments, terpenes such as limonene are used.

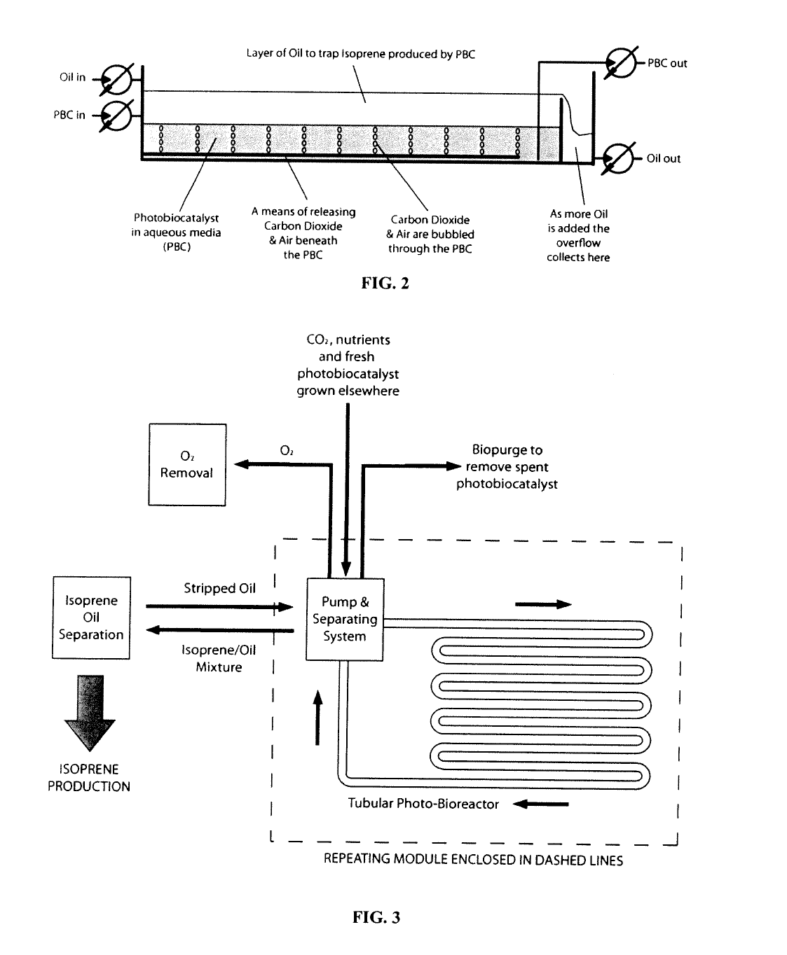

In some embodiments, the rate of flow of the photobiocatalyst contained in aqueous media, and the oil used to capture isoprene, are controlled independently. FIG. 2 is a cross-sectional view of the photobioreactor portion of the exemplary photo-biochemical reactor shown in FIG. 1. The aqueous media containing the photobiocatalyst is fed into the open vessel from a remote photobioreactor. A mixture of air to provide required nitrogen and carbon dioxide is bubbled through the open vessel of a nearly stagnant photobiocatalyst. The photobiocatalyst is periodically removed from the open vessel and replenished with fresh photobiocatalyst. The photobiocatalyst in aqueous media is separated from the atmosphere by a layer of oil, which is immiscible with aqueous solutions, captures isoprene produced by the photobiocatalyst, and allows oxygen to pass through. In some embodiments, oil added to the open vessel sits on top of the aqueous phase. A flow of oil may be maintained by adding oil devoid of isoprene at one end and collecting the overflow of oil loaded with isoprene at the other end of the open vessel. The oil loaded with isoprene is directed to a means for separating the oil from the isoprene. In some embodiments, the flow of oil over the aqueous media containing photobiocatalyst is at a higher rate than the underlying aqueous phase (FIG. 2).

In some embodiments, the aqueous media containing the photobiocatalyst and the absorbent are delivered into the vessel, e.g. covered or open, from one end and recovered from the other end of the vessel, e.g. covered or open, using independent systems. In the vessel, e.g. covered or open, the photobiocatalyst consumes water and carbon dioxide dissolved in the aqueous medium to produce isoprene and oxygen. The isoprene produced dissolves in the oil layer covering the aqueous media and the oxygen produced passes freely through the oil and pass into the atmosphere. The mixture of aqueous media containing the photobiocatalyst passes through the pumping system. Additional carbon dioxide, nutrients and water necessary to support the photobiocatalyst are added and photocatalyst that is no longer viable is removed. As the oil is piped to a separating system, oil loaded with isoprene is separated from the aqueous media and photobiocatalyst mixture and is sent to a means for separating isoprene and oil. Oil stripped of isoprene is returned to the aqueous media and photobiocatalyst mixture from the means for separating isoprene and oil (FIG. 1).

In some embodiments, the system of the invention comprises a photo-biochemical reactor comprising an open vessel photobioreactor, a separating system, a means for adding carbon dioxide, nutrients, water and fresh photobiocatalyst, a means for separating isoprene from oil loaded with isoprene, a means for returning stripped oil, and a means for capturing stripped isoprene from the oil, as described herein. In other embodiments, a series of long tubes constructed of polymer film such as polyethylene, filled with aqueous media and installed at a slight incline along the length which provides the impetus for gases or low density, immiscible, liquids, such as isoprene to travel along the length of the tube to an exit point where the fluids pass into a layer of oil which acts as an absorbent, capturing the isoprene and allowing any gases, including oxygen, to pass through, a separating system, a means for adding carbon dioxide, nutrients, water and fresh photobiocatalyst, a means for separating isoprene from oil loaded with isoprene, a means for returning stripped oil, and a means for capturing stripped isoprene from the oil, as described herein. In some embodiments, the system comprises one or more of the above-described components of a photo-biochemical reactor, and optionally comprises one or all of these components.

The means for adding carbon dioxide, nutrients, water and fresh photobiocatalyst can be a tube or pipe which communicates with an inlet of the bioreactor, or other unit operations known in the art. In some embodiments, a pump is employed in the system.

The means for separating isoprene from oil loaded with isoprene can be a tank (a separation tank), or a column where the loaded oil is placed. In some embodiments, the system is also equipped with a heating source to heat the oil to a temperature above the boiling point of isoprene but below the boiling point of the oil.

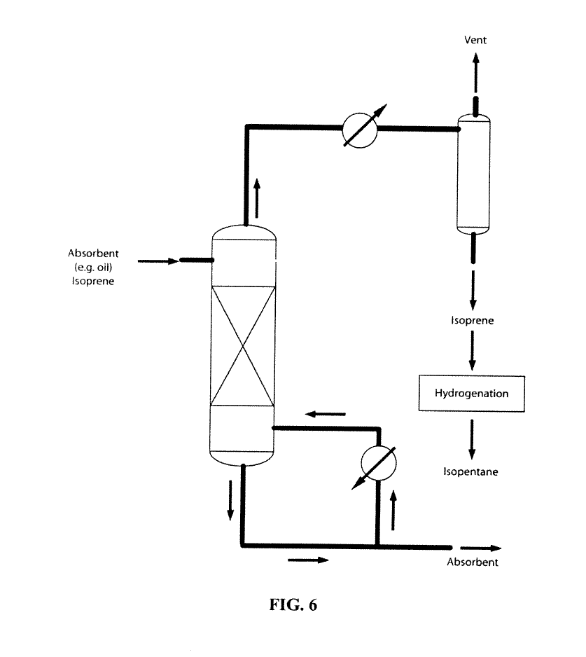

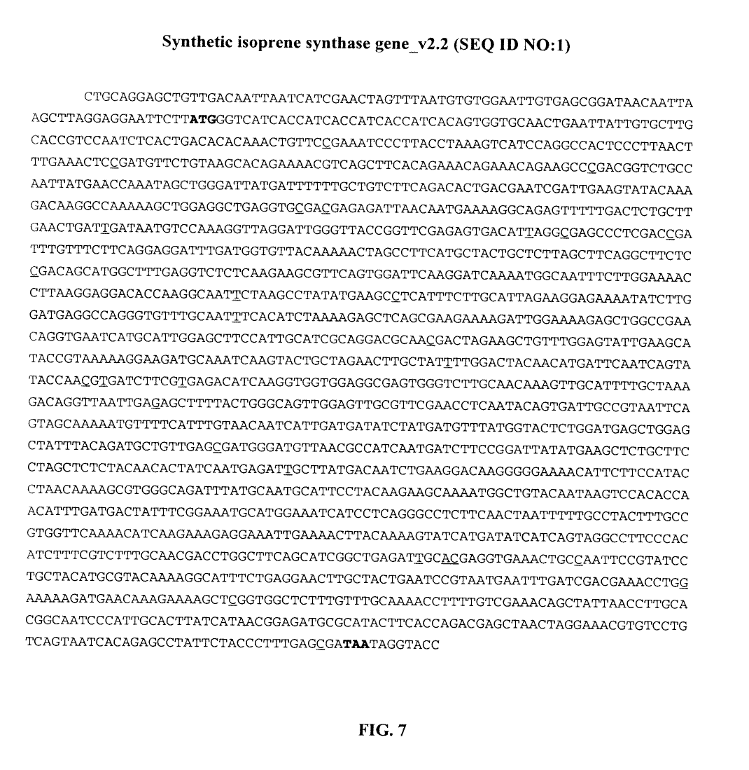

In some embodiments, the system comprises a stripping system. The term “stripping,” as used herein, refers to the removal by evaporation of the more volatile components from liquid, a common process applied in the chemical industry. FIG. 5 illustrates an Isoprene Stripping system. In this system, a liquid comprised of an oil that is significantly less volatile than isoprene, containing a dissolved quantity of isoprene, is fed to a stripping column. In the column, the liquid is distributed over high surface area structures and exposed to heat. The more volatile isoprene leaves the top of the column as a vapor. It enters a heat exchanger where it is condensed and sent to storage. The oil leaves the bottom of the column where a portion is heated and returned the stripping column. The remaining oil that is substantially free of isoprene is then sent to storage and can be either fed back into the system or discarded.

Other methods are known in the art for capturing organic products from a bacterial culture which can be used in the present invention to capture the volatile carbonaceous chemical using an oil. (Janikowski T B et. al., Appl Microbio Biotechnology, 59:368-376 (2000); Newman J D, et al., Biotechnology and Bioengineering, 95:684-691 (2006); Daugulis A, Trends in Biotechnology 19:457-462 (2001), all incorporated herein by reference.

The means for returning stripped oil can be a tube or pipe connected to an outlet of the bioreactor, or other unit operations known in the art. In some embodiments, a pump is employed in the system.

The means for capturing stripped isoprene from the oil can be a tube or pipe that connects to an outlet of the separation tank, or the like.

In some embodiments, the aqueous media containing the photobiocatalyst and oil are pumped through the tubing and through a separating system. The photobiocatalyst is grown in one photobioreactor and then fed into the tubular photobioreactor portion of the photo-biochemical reactor. As the mixture of aqueous media containing the photobiocatalyst and oil passes through the separating system, additional carbon dioxide, nutrients and water necessary to support the photobiocatalyst are added and photobiocatalyst that is no longer viable is removed. As the mixture of aqueous media containing the photobiocatalyst and oil passes through the separating system, oxygen produced as a byproduct of photosynthesis is safely removed in a manner that prevents it being a flammable or explosive hazard. As the mixture of media containing the photobiocatalyst and oil passes through the separating system, oil loaded with isoprene is separated from the aqueous media and photobiocatalyst mixture and sent to a means for separating isoprene and oil. The mixture of aqueous media containing the photobiocatalyst and oil passes through the separating system, oil stripped of isoprene is returned to the aqueous media and photobiocatalyst mixture from the means for separating isoprene and oil. One embodiment of this system, also referred to as a photobioreactor, is illustrated in (FIG. 3).

In some embodiments, the system of the invention comprises a photo-biochemical reactor comprising a photobioreactor comprising a plurality of long tubes constructed of polymer film such as polyethylene filled with aqueous media and installed at a slight incline, a separating system, a means for adding carbon dioxide, water, nutrients, and fresh photobiocatalyst, a means for removing oxygen and a means for separating isoprene from oil loaded with isoprene, a means for returning stripped oil, and a means for capturing stripped isoprene from the oil. In some embodiments, the system of the invention comprises of at least one of these components of a photo-biochemical reactor, and optionally comprises more than one of any or all of these components.

### Isoprene Product Separation and Capture System to Form Part of the Photo-biochemical Reactor

In another aspect, the present invention provides a product capture and collection system. The product capture system permits isoprene product capture, provides oxygen export to limit formation of flammable hydrocarbon mixtures, and prevents peroxide formation.

In some embodiments, the capture and collection system is connected to a pumping and separation system. In one embodiment, the photobioreactor comprises a plurality of long tubes constructed of polymer film such as polyethylene, filled with aqueous media and installed at a slight incline along the length which provides the impetus for gases or low density, immiscible, liquids, such as isoprene to travel along the length of the tube to an exit point where the fluids pass into a layer of oil which acts as an absorbent, capturing the isoprene and allowing any gases, including oxygen to pass through (FIGS. 4A and 4B). The oil loaded with isoprene is then sent to a means of separating the oil and the isoprene, and the isoprene is collected therefrom.

In other embodiments, the oil phase is kept in a separate compartment or vessel dimensioned and configured to communicate with the photobioreactor in a manner which permits the passage of gas, e.g. oxygen and/or volatile carbon product, e.g. isoprene, from the photobioreactor through and to the oil phase, respectively.

In another embodiment, the photobioreactor is an enclosed system that prevents the release of oxygen, and the oil and photobiocatalyst are mixed together as they both flow through the photo-biochemical reactor. In this embodiment, the photo-biochemical reactor has a capture and collection system comprising a vessel in which an aqueous phase containing the photobiocatalyst and the aqueous media settles at the bottom of the vessel, an oil, in which isoprene could dissolve, but which is immiscible with water, loaded with isoprene would sit on top of the aqueous phase, and a gaseous phase comprising largely oxygen produced by photosynthesis would be above both the aqueous and oil phases (FIG. 5).

In some embodiments, the aqueous phase containing the photobiocatalyst is pumped back to the photobioreactor portion of the photo-biochemical reactor, whether the photobioreactor portion is an open vessel or an enclosed system. On the way to the photobioreactor portion, a purge is taken to remove dead photobiocatalyst. Water, aqueous media and photobiocatalyst are added to the aqueous phase to maintain an optimal amount and concentration of photobiocatalyst. Oil stripped of the product isoprene and/or fresh oil are added to the aqueous phase. Carbon dioxide, either pure, from the atmosphere, or from a flue of another facility producing carbon dioxide, is bubbled into the aqueous media containing the photobiocatalyst. The oil phase, rich in isoprene in the column, is pumped to another system for stripping the isoprene therefrom. The gaseous phase, which is largely oxygen, is sent to another system where the oxygen is either vented, compressed, or incinerated (also FIG. 5).

In some embodiments of this invention, isoprene is produced, isolated, and used in fuel either as is, or after further processing (e.g., hydrogenation).

In another aspect, the invention provides a carbonaceous chemical (e.g., isoprene) produced by the methods described herein. In another aspect, the invention provides a hydrocarbon fuel (e.g., isopentane) produced by hydrogenating said carbonaceous chemical.

Options for Incorporating Isoprene into Transportation Fuels

Isoprene (C₅H₈, 2 methyl 1,3 butadiene) is a low molecular weight (68), single branched hydrocarbon that contains one set of conjugated double bonds. It has a normal boiling point of 93° F. (34° C.) and a specific gravity of 0.686. Isoprene is present in small amounts in much of the gasoline currently sold in the US. It is a minor product of the thermal and catalytic cracking of heavier oils to increase the production of gasoline and diesel fuel. Isoprene, like other diolefins present in gasoline, can polymerize and form gums that can foul carburetors and fuel injectors. Gasoline typically contains additives that inhibit these polymerization reactions. Some amount of isoprene could be blended directly into the ˜2.5 billion lbs per day of US gasoline consumption. It has a relatively high Reid vapor pressure (RVP), which would limit the amount that could be blended into gasoline. Gasoline is currently blended to an RVP limit by the addition of relatively low value light hydrocarbons. The value of isoprene as a direct gasoline blending component would be a function of the value of other high RVP components it might replace. The amount of polymerization inhibitor that is used might also have to be increased, but this is used in small amounts and would have minimal impact on the value of isoprene as a direct blending component.

The use of a polymerization inhibitor could be reduced by the partial hydrogenation of isoprene to an equilibrium mixture of branched C5 olefins. Partial hydrogenation is a low severity process that is currently used in many refineries to remove sulfur from various gasoline blending components. Some amount of isoprene could be added to the feed of existing gasoline hydrotreaters. The primary operating cost for these units is the hydrogen that is consumed in the hydrotreating reactions. Mild hydrogenation would consume 1 mole of hydrogen per mole of Isoprene. Conversion of the diolefin to an olefin would decrease its density while slightly increasing its RVP and octane. Since gasoline is sold by volume and not weight, this density decrease actually increases the amount of material that is available for sale. This increase in volume would compensate for the cost of the hydrogen.

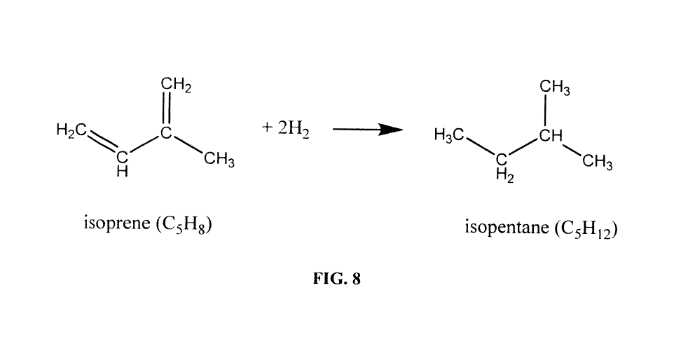

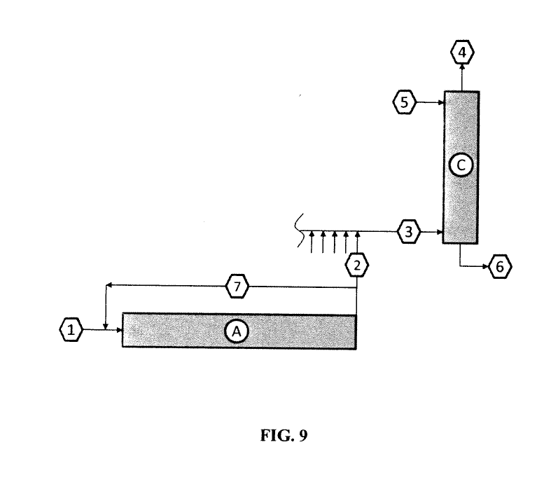

Hydrogenation is the chemical reaction that results from the addition of hydrogen (H₂). The process is usually employed to reduce or saturate organic compounds including isoprene. The process typically constitutes the addition of pairs of hydrogen atoms to a molecule. Catalysts are required for the reaction to be usable; non-catalytic hydrogenation takes place only at very high temperatures. Hydrogen adds to double and triple bonds in hydrocarbons. Hydrogenation of isoprene produces isopentane also known as C₅H₁₂, also called methylbutane or 2-methylbutane, a branched-chain alkane with five carbon atoms (FIG. 8). Isopentane is an extremely volatile and extremely flammable liquid at room temperature and pressure. The normal boiling point is just a few degrees above room temperature and isopentane will readily boil and evaporate away on a warm day. As such it is a common component of gasoline.

In some embodiments of the invention, the isoprene separated from the oil is hydrogenated to produce compounds such as isopentane, which is used in the production of fuels including gasoline. The isoprene will be combined with a source of hydrogen, in the presence of a catalyst that binds to both isoprene and hydrogen. Exemplary catalysts useful for this purpose include platinum group metals, particularly platinum, palladium, rhodium, and ruthenium, and non-precious metal catalysts, especially those based on nickel (such as Raney nickel and Urushibara nickel). The isopentane produced from photosynthetically produced isoprene is a renewable source of liquid fuel. In some embodiments of this invention, other terpenes, in addition to the hemiterpene isoprene, are produced, isolated and used in fuel either as they are, or after further processing (e.g., hydrogenation). Terpenes are classified by the number of terpene units in the molecule; a prefix in the name indicates the number of terpene units needed to assemble the molecule. Hemiterpenes consist of a single isoprene unit. Isoprene itself is considered the only hemiterpene, but oxygen-containing derivatives such as prenol and isovaleric acid are hemiterpenoids. Monoterpenes consist of two isoprene units and have the molecular formula C₁₀H₁₆. Examples of monoterpenes include, but are not limited to, geraniol, limonene and terpineol. Sesquiterpenes consist of three isoprene units and have the molecular formula C₁₅H₂₄. Examples of sesquiterpenes include, but are not limited to, farnesenes, farnesol. The sesqui-prefix means one and a half. Diterpenes are composed for four isoprene units and have the molecular formula C₂₀H₃₂. They derive from geranylgeranyl pyrophosphate. Terpenes containing larger numbers of terpene units exist, but will not be efficiently produced and isolated by the system described here.

There are various conventional refining processes that could further upgrade isoprene for inclusion in gasoline without the RVP penalty, or for inclusion in jet fuel or diesel fuel. Generally, these processes would first require the partial hydrogenation of isoprene to mixed isopentenes.

Many US refineries have alkylation units that react isobutene with butylenes and sometimes propylene to produce alkylate which is a mixture of iso-octanes (from butylenes) and iso-heptanes (from propylene). Alkylate is a valuable gasoline blending component because of its high octane and low RVP, sulfur and benzene. Small amounts (up to 5% of the total feed) of mixed pentenes are also currently fed to many alkylation units. The pentenes produce a slightly lower octane alkylate, but alkylation significantly lowers the RVP of the pentene feed. At 5% of the feed, current US alkylation capacity could absorb over 5 million lbs per day of isoprene (hydrogenated to pentenes). Many commercial alkylation units already mildly hydrotreat their feeds to covert diolefins of olefins. This units could accept Isoprene with little difficulty.

Less common though commercially viable processes for upgrading isoprene again include conversion of isoprene to mixed isopentenes as a first step. Catalytic polymerization of propylene to mixed hexenes and nonenes is currently practiced in many refineries. This process would also be viable for reacting pentenes to higher molecular weight olefins. Process conditions can be adjusted to produce a dimer (C10 olefin) or trimer (C15 Olefin). Current US polymerization capacity for gasoline production exceeds 10 million lbs per day.

Pentene dimers (decenes) could be blended directly into gasoline. Small amounts of the dimers and trimers could also be blended directly into diesel fuel. Hydrogenation of the dimer and/or trimer to alkanes would allow blending of high volumes (up to 50% or more) of these materials into either diesel fuel or jet fuel. The hydrogenated products would have excellent properties as jet fuel or diesel fuel components.

Although no commercial units currently are operational, the MTG process, developed and commercialized by Mobil Technology Company in the 1980's could also convert the pentenes (partially hydrogenated isoprene) into a gasoline blending component.

Also provided herein is a fuel product comprising the carbonaceous chemical, e.g isoprene, or the hydrocarbon fuel, e.g. isopentane, as described above, and a fuel component. In some instances, the fuel component is a blending fuel which may be fossil fuel, gasoline, diesel, ethanol, jet fuel, or any combination thereof. In some embodiments, the fuel component is a fuel additive which may be MTBE, an anti-oxidant, an antistatic agent, a corrosion inhibitor, and any combination thereof. In some instances, the carbonaceous chemical or hydrocarbon fuel comprises an isoprene unit. In another instance the carbonaceous chemical or hydrocarbon fuel comprises a terpene. In other instances, the hydrogen and carbon atoms are at least 90% of the weight of the composition component. In still other instances, the hydrogen and carbon atoms are at least 95% or at least 99% of the weight of the composition component. For some fuel products, the carbonaceous chemical is terpene. In some instances, the carbonaceous chemical or hydrocarbon fuel is a liquid.

The isoprene produced by the method of the present invention is, in one embodiment, at least about 90% pure, e.g., at least about 92%, 94%, 96% or at least about 98% pure. In various embodiments, the invention provides isoprene at least about 98%, at least about 98.5%, at least about 99% or at least about 99.5% pure. In exemplary embodiments, the invention provides isoprene that is at least about 95% pure, e.g., at least about 96%, 97%, 98% or at least about 99% pure.

A fuel product as described herein may be a product generated by blending a carbonaceous chemical or hydrocarbon fuel, and a fuel component. For example, a carbonaceous chemical or hydrocarbon fuel as described herein can be blended with a fuel component prior to refining (for example, cracking) in order to generate a fuel product as described herein. A fuel component, as described, can be a fossil fuel, or a mixing blend for generating a fuel product. For example, a mixture for fuel blending may be a hydrocarbon mixture that is suitable for blending with another hydrocarbon mixture to generate a fuel product. For example, a mixture of light alkanes may not have a certain octane number to be suitable for a type of fuel, however, it can be blended with a high octane mixture to generate a fuel product.

In some instances, the carbonaceous chemical/hydrocarbon fuel or fuel component alone are not suitable as a fuel product, however, when combined, they comprise a fuel product. In other instances, either the carbonaceous chemical/hydrocarbon fuel or the fuel component or both individual are suitable as a fuel product. In yet other instances, the fuel component is an existing petroleum product, such as gasoline or jet fuel. In yet other instances, the fuel component is derived from a renewable resource, such as bioethanol, biodiesel, biogasoline, and the like.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the description and drawings are by way of example only.

The present invention is illustrated by the following examples, which are not intended to be limiting in any way.

### EXAMPLES

### Example 1

### Synthesizing a Gene for Isoprene Synthase (v2.2)

A cDNA clone for isoprene synthase was cloned from the poplar (Miller, et al. (2001). Planta 213, 483-487). Expression of this foreign gene in E. coli, and production of isoprene from the recombinant organism, has also been demonstrated (Miller, et al. (2001). Planta 213, 483-487). The class of enzymes to which isoprene synthase belongs, terpene cyclases, has been relatively well-studied, e.g. the determinations of the 3D structures of the homologs 5-epi-aristolochene synthase (Starks, et al. (1997) Science 277, 1815-1820) and bornyl diphosphate synthase (Whittington, et al. (2002) Proc. Natl. Acad. Sci. U.S.A. 99, 15375-14380). The structures are used to aid protein engineering experiments for optimizing the enzyme.

A gene encoding isoprene synthase is synthesized using the technology known in the art and modified to: (a) provide optimized codons for expression in T. elongatus, and (b) remove or insert certain restriction sites (e.g., sites recognized by the T. elongatus restriction enzyme system (Iwai, et al. (2004) Plant Cell Physiol. 45, 171-175) or sites necessary for molecular biological manipulations), and/or mutate the amino acid sequence to favorably alter the physical properties of the isoprene synthase protein itself (e.g, provide more thermostability, etc.). (FIG. 6)

### Example 2

### Engineering Thermosynechococcus elongatus BP-1 to Express Isoprene Synthase

Thermosynechococcus elongatus BP-1 is a particularly favorable cyanobacteria strain with which to assess expression of isoprene synthase. There has been extensive characterization of the photosynthetic machinery of this microorganism (Rutherford, and Boussac, (2004) Clefs CEA 49, 86-92) and the complete DNA sequence of its genome has been determined and annotated (Nakamura, et al. (2002). DNA Res. 9, 123-130). Moreover, transformation with plasmids via electroporation and expression of foreign genes has been demonstrated (Iwai, et al. (2004) Plant Cell Physiol. 45, 171-175). Thus, all the basic tools exist for metabolic engineering with T. elongatus. T. elongatus also has an optimal growth temperature of 55° C., more than 20° above the boiling point for isoprene, meaning that the kinetics of isoprene volatilization from the living cells should be extremely favorable, “pulling” the reaction forward.

The synthetic isoprene synthase gene is inserted into a vector behind a promoter that is transcriptionally active in T. elongatus; such vector can also contain markers allowing for selection of positive transformants in T. elongatus (Iwai, et al. (2004) Plant Cell Physiol. 45, 171-175).

### Example 3

### Test Cultures

Test cultures of the recombinant strain on minimal medium containing trace elements but no carbon source (apart from CO₂) are conducted to establish photosynthetic production of isoprene from carbon dioxide, according to three criteria: (i) detection of levels of off-gassed isoprene significantly higher than those found in the off-gas from non-recombinant control cells; (ii) demonstration of the light-inducibility and light-dependence of isoprene synthesis; and (iii) demonstration that the isoprene can be isotopically labeled by culturing the recombinant organism in the presence of ¹³CO₂.

### Example 4

### Synthesizing and Expressing a Gene for Isoprene Synthase (v2.2.1) in Cyanobacteria and Detection of Culture-derived Isoprene

Briefly, a synthetic isoprene synthase gene, cloned into the vector pUC57 as a PstI/KpnI fragment (FIG. 6), was constructed. Underlined are selected alterations of the poplar enzyme coding sequence to remove certain restriction enzyme sites and to substitute rare codons (based on T. elongatus BP-1 codon usage) for more common ones, as well as several other base changes. The start (ATG) and stop (TAA) codons are indicated in bold.

To facilitate detection and/or purification of the recombinant enzyme a His₆ tag coding sequence has been fused 5′ to the enzyme coding sequence immediately after the start codon. This tag facilitates detection of the recombinant enzyme in situ on SDS gels using InVision™ stain (Invitrogen, Inc.) and/or purification of the recombinant enzyme from cell extracts using immobilized metal affinity chromatography. Immediately upstream of the start codon is a ribosome binding site sequence preceded by a tach promoter (de Boer, et al. (1983). Proc. Natl. Acad. Sci. U. S. A 80, 21-25) a unique EcoRI site between the start codon and the ribosome binding site allows the fragment containing these upstream control sequences to be separated from the coding sequences, if desired. This coding sequence, with or without further “tailoring” of the ends, was placed under control of the Anabaena pNir promoter which drives expression of the gene encoding nitrate reductase (Desplancq, et al. (2005). Biotechniques 39, 405-411).

Desplancq et al, includes the construction of p505 with the pNir promoter driving the transcription of the hetR gene. We cut out the hetr gene with EcoRI and BamHI and replaced it with the amplified isoprene synthase gene. The primers used to amplify the isoprene synthesas gene with the EcoRI and BamHI sites attached were the following:
EccoRI-5′-CAGGAATTCATGGCAACTGAATTA′TTGTGCTTG-3′ (SEQ ID NO: 2)
BamHI-5′-CAAGGATCCTTATCGCTCAAAGGGTAGAA-3′ (SEQ ID NO: 3)

This promoter is OFF in cells growing on ammonia as nitrogen source but ON strongly in cells growing on nitrate. For engineering applications this promoter is useful for short-term batch applications, but not generally applicable because the nitrate is consumed during growth.

This construct was maintained as a plasmid in Anabaena 7120 by continuous selection in neomycin at 25° C. After 10 days visible colonies were picked and transferred to liquid medium, either BG-11 or BG-11 without combined nitrogen, but with neomycin. After ten days of growth, nitrate was added to the BG-11₀ culture to induce transcription of isoprene synthase. The isoprene synthase transcript was detected by RT-PCR. After several days, the cell culture temperature was increased to 37° C. to permit conversion of any isoprene produced to the gas phase.

Test fermentations of the transformed strains on minimal medium containing trace elements but no carbon source (apart from CO₂) were conducted to establish photosynthetic production of isoprene from carbon dioxide. Successful expression of the isoprene synthase gene in the recombinant cyanobacteria was demonstrated by the following outcomes:

(a) Transformed cells by comparison with appropriate control strains were demonstrated to have the ability to transcribe IS mRNA by PCR.

(b) Biosynthetic production of isoprene from recombinant cell cultures was demonstrated by gas chromatography/mass spectrometry using protocols developed to detect isoprene given off by bacterial cultures (Kuzma, et al. (1995) Curr. Microbiol. 30, 97-103—see below). Levels of outgassed isoprene were higher than those found in the outgas from non-recombinant control cells. The calculated yield of isoprene under the conditions listed are 25 micrograms per liter per 30 min of a culture at OD700=0.23.

The isoprene product was harvested by passing the off-gas through a condenser and then washing it out of the condenser with the solvent dichloroethane. The (concentrated) DCE solution is injected into the GC for quantitation. Assays of isoprene produced by bacterial cell cultures were conducted as described in Kuzma, et al. (1995) Curr. Microbiol. 30, 97-103.

Identification of isoprene by gas chromatography-mass spectrometry (GC-MS). Bacteria are inoculated into 10 ml of rich media and grown to an A₆₀₀ of approximately 1.5. Then, 2 ml of culture are incubated in 4.8 ml glass vials sealed with Teflon-lined septa for approximately 6 hours. The sample headspace (1.2 ml) is collected in a nickel loop packed with glass beads immersed in liquid argon (−186° C.). The loop is subsequently heated to 150° C. and injected into a DB-1 column (30 m long, 0.25 mm diameter, 1 μm film thickness) (J & W Scientific, Folsom, Calif.) connected to a 5971A Hewlett Packard mass selective detector (electron ionization, operated in total ion mode) or an equivalent instrument. The temperature program for each GC-MS run includes a 1 minute hold at −65° C. followed by a warming rate of 4° C. per minute. Helium carrier gas and a flow rate of approximately 0.7 ml min⁻¹ are used. This system is described in more detail in (Cicerone, et al. (1988). J. Geophys. Res. 93, 3745-3749). For the positive identification of bacterial isoprene production, peak retention times and mass spectra obtained from bacterial headspace are compared with the retention time and mass spectrum of an authentic isoprene standard. The headspace from a “vector only” recombinant control culture should be run as a negative control.

Routine isoprene assays. Bacterial strains are grown to an A₆₀₀ ranging from 1.0 to 6.0. Two ml of culture are incubated in sealed vials at an appropriate temperature with shaking for approximately 3 hours; headspace is analyzed with a gas chromatography (GC) system that is highly sensitive to isoprene (for example, see (Greenberg, et al. (1993). Atmos. Environ. 27A, 2689-2692), and (Silver and Fall (1991) Plant Physiol. 97, 1588-1591)). The system is operated isothermally (85° C.) with an n-octane/porasil C column (Alltech Associates, Inc., Deerfield, Ill.) and is coupled to an RGD2 mercuric oxide reduction gas detector (Trace Analytical, Menlo Park, Calif.) or its equivalent. Isoprene elutes at 3.6 minutes. Isoprene production rates (nmol g⁻¹ h⁻¹) can be calculated as follows: GC area units are converted to nmol isoprene via a standard isoprene concentration calibration curve; A₆₀₀ values for the samples are taken and converted to grams of cells (g) by obtaining wet weights for cell cultures with a known A₆₀₀. Two to five separate measurements are taken and averaged for each assay point. Negative controls are as above.

### Example 5

### System for Hydrocarbon Production and Capture

Referring to FIG. 9, an integrated process for the production and recovery of isoprene. A series of photobioreactors (A) containing photosynthetic microbes suspended in water and provided with adequate nutrients are fed a gas mixture (1) containing carbon dioxide, with or without inert gases such as nitrogen, which is converted either fully or partially to isoprene. The quantity and composition of inert gases can be varied to utilize the lowest cost source of carbon dioxide and/or to provide sufficient gas flow to optimize the production of isoprene and/or to ensure that the gas mixtures in the system are non-flammable.

In the exemplary system of FIG. 9, product isoprene along with by-product oxygen, any unreacted carbon dioxide, and any inert gases, passes out of the each individual photobioreactor (2). In order to optimize isoprene production, a portion of the vapor exiting the photobioreactor (7) may be recycled and mixed with the feed gas mixture (1). The photobioreactor exit vapor stream (2) is combined with the vapor streams leaving other photobioreactors, and is fed (3) to the product absorber (C). The product absorber comprises a system for the intimate contact of liquid organic absorbent with the incoming vapor stream to effect the nearly complete recovery of the isoprene contained therein yielding an exit gas (4) essentially free of isoprene. Liquid organic absorbent containing little or no isoprene (5) is fed to the product absorber. As the liquid organic absorbent passes down through the product absorber it intimately contacts the vapor stream absorbing the product isoprene. Liquid organic absorbent (6) containing the product isoprene is then removed from the product absorber and sent to the product purification system.

Vapor compositions for the streams indicated in FIG. 9 for this exemplary embodiment of the invention are provided below. The numbers at the top of the columns below correspond to numbers in hexagons on the diagram of FIG. 9.

| | Stream # on FIG. 9 | | | |
|---|---|---|---|---|
| | (1) | (2) | (3) | (4) |
| Component | Gas/vapor stream compositions % by volume | | | |
| Carbon dioxide | 15.0% | 11.8% | 11.8% | 11.8% |
| Isoprene | 0.0% | 0.6% | 0.6% | 0.0% |
| Nitrogen | 78.0% | 76.5% | 76.5% | 77.0% |
| Oxygen | 2.0% | 6.1% | 6.1% | 6.1% |
| Water | 5.0% | 5.0% | 5.0% | 5.0% |

### Example 6

### System for Hydrocarbon Production and Capture

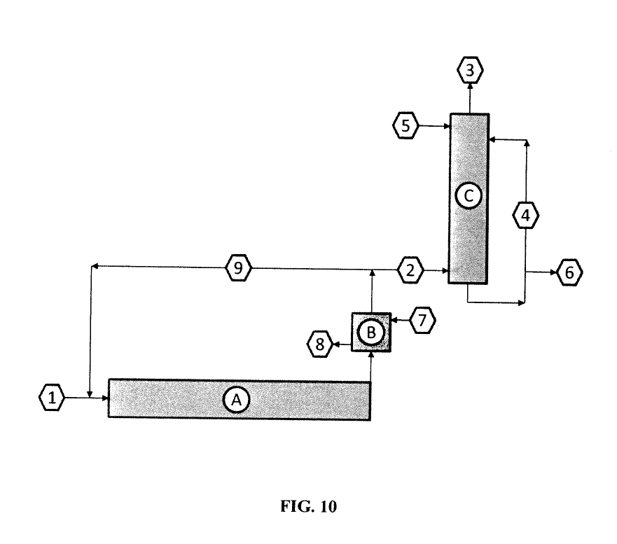

Referring to FIG. 10, the system may comprise multiple product absorbers arranged in series, e.g. an initial product absorber (B) and a secondary product absorber (C). Illustrating the respective function of the various system components using isoprene production as an example, the product isoprene, in either gaseous or liquid form, along with by-product oxygen, any unreacted carbon dioxide, and any inert gases, passes out of the each individual photobioreactor through an initial product absorber (B) which is comprised of a volume of liquid organic absorbent, selected based on its affinity for isoprene and its compatibility with other system components and conditions inside a physical structure, with or without internal structures to enhance mass transfer. The initial product absorber of FIG. 10 essentially serves to capture a portion of the isoprene produced, including any liquid isoprene produced, as well as help ensure that the gas stream exiting the initial product absorber (2) is non-flammable. Liquid organic absorbent (7), containing low levels of isoprene, is added to the initial product absorber either continuously or intermittently and liquid organic absorbent containing isoprene (8) is withdrawn from the initial product absorber and either utilized elsewhere in the product recovery process or sent directly to the isoprene stripping unit to recover the product isoprene and yield liquid product absorbent which is returned to the product recovery system.

In the exemplary system of FIG. 10, the vapor stream leaving the initial product absorbers (2) is collected and fed to a secondary product absorber (C). In order to optimize the isoprene production and recovery process, a portion of the vapor stream leaving the initial product absorber (9) may be recycled back to the photobioreactor. The secondary product absorber comprises a system for the intimate contact of liquid organic absorbent with the incoming vapor stream to effect the nearly complete recovery of the isoprene contained therein yielding an exit gas (3) essentially free of isoprene. Liquid organic absorbent containing little or no isoprene (5) is fed to the secondary product absorber. Liquid organic absorbent is circulated (4) over the secondary product absorber to effect the capture of isoprene from the vapor phase and the net quantity of isoprene captured is removed by sending a stream of liquid organic absorbent (6) from the circulation system, either intermittently or continuously, to be utilized elsewhere in the product recovery process or sent directly to the isoprene stripping unit to recover the product isoprene and yield liquid product absorbent which is returned to the product recovery system.

Referring to FIG. 10, providing an alternative embodiment of the invention, vapor compositions for the streams indicated therein are provided below. The numbers at the top of the columns below correspond to numbers in hexagons on the diagram of FIG. 10.

| Gas/Vapor Stream | | | |
|---|---|---|---|
| % by volume | 1 | 2 | 3 |

Example 6A

| | | | |
|---|---|---|---|
| Carbon Dioxide | 15.0% | 7.2% | 7.3% |
| Isoprene | 0.0% | 1.4% | 0.0% |
| Nitrogen | 85.0% | 81.3% | 82.5% |
| Oxygen | 0.0% | 10.0% | 10.2% |

Example 6B

| | | | |
|---|---|---|---|
| Carbon Dioxide | 15.0% | 4.7% | 4.7% |
| Isoprene | 0.0% | 0.9% | 0.0% |
| Nitrogen | 78.0% | 80.8% | 81.6% |
| Oxygen | 2.0% | 8.6% | 8.7% |
| Water | 5.0% | 5.0% | 5.0% |
| | 100.0% | 100.0% | 100.0% |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. All publications and patents cited herein are incorporated by reference.

1. A method for producing a volatile carbonaceous chemical, the method comprising the steps of:
(a) maintaining said photobiocatalyst in stationary phase under a first culture condition sufficient for said photobiocatalyst to convert a feedstock comprising carbon dioxide into said carbonaceous chemical through photosynthesis;
(b) collecting said volatile carbonaceous chemical in an absorbent medium essentially miscible with said volatile carbonaceous chemical; and
(c) separating said volatile carbonaceous chemical from said absorbent medium.

2. The method of claim 1, further comprising the step of:
(d) hydrogenating said volatile carbonaceous chemical.

3. The method of claim 1, further comprising growing a photosynthetic microorganism under a second culture condition to produce a photobiocatalyst.

4. The method of claim 1, wherein said photosynthetic microorganism is genetically engineered to increase the carbon flux from photo-synthetically fixed carbon dioxide to produce said volatile carbonaceous chemical.

5. The method of claim 1, wherein said photosynthetic microorganism is genetically modified to maintain constant cell density and cell size such that most energy is expended in the biosynthesis of said volatile carbonaceous chemical in response to a controllable signal.

6. The method of claim 1, wherein said volatile carbonaceous chemical comprises terpene.

7. The method of claim 1, wherein said volatile carbonaceous chemical comprises hemiterpene.

8. The method of claim 7, wherein said hemiterpene is isoprene.

9. The method of claim 1, wherein said photosynthetic microorganism is a cyanobacterium comprising a transgenic terpene synthase gene.

10. The method of claim 9, wherein said terpene synthase is an isoprene synthase gene derived from poplar or kudzu.

11. The method of claim 1, wherein said first culture condition is favorable for said photosynthetic microorganism to divide.

12. The method of claim 1, wherein said second culture condition is not favorable for said photosynthetic microorganism to divide.

13. The method of claim 1, wherein said second culture condition optimizes production of said volatile carbonaceous chemical.

14. The method of claim 1, wherein said photosynthetic microorganism is a cyanobacterium that can be genetically manipulated, has long division time, does not require nitrate to grow, grows in salt water, and tolerates a temperature above 34° C.

15. The method of claim 1, wherein said photobiocatalyst grows in said photobioreactor, and does not grow, or grows slowly, when used as a catalyst in said photo-biochemical reactor for the production of said volatile carbonaceous chemical.

16. The method of claim 5, wherein said controllable signal is depriving cells of key nutrients required for the generation of biomass, changing the pH range to disfavor the generation of biomass, or changing the temperature.

17. The method of claim 5, wherein said genetic modification is the introduction of a gene critical for inhibiting key hydrocarbon formation under the control of an inducible or constitutive promoter, or a regulatory sequence.

18. The method of claim 17, wherein said introduced gene is under the control of a pNir promoter that can be activated by the addition of nitrate ions.

19. The method of claim 17, wherein said introduced gene is under the control of a pPetE promoter that can be activated by the addition of copper ions.

20. The method of claim 1, wherein said photobiocatalyst comprises an isolated chloroplast.

21. The method of claim 1, wherein said absorbent medium is an organic substance.

22. The method of claim 1, wherein said absorbent medium is an isoparaffinic fluid.

23. The method of claim 1, wherein step (a) occurs in a photobioreactor.

24. The method of claim 1, wherein step (b) occurs in a photo-biochemical reactor.

25. The method of claim 24, wherein said photo-biochemical reactor comprises a plurality of tubes filled with an aqueous medium and installed on a slight incline along the length which provides the impetus for gases or low density immiscible liquids to travel along the length of said tubes to an exit point where said gases or liquids pass into said absorbent medium.

26. The method of claim 1, wherein said separating step comprising removing said volatile carbonaceous chemical from said absorbent medium by heating.

27. A system comprising:
(a) a photo-biochemical reactor for producing a mixture of gases or low density immiscible liquids, said photo-biochemical reactor comprising:
(i) a plurality of tubes filled with an aqueous medium and installed on a slight incline along the length which provides the impetus for gases or low density immiscible liquids to travel along the length of said tubes to an exit point where said gases or liquids pass into a layer of absorbent medium;
(ii) a photobiocatalyst; and
(iii) optionally, a cover which allows photosynthesis but prevents heating to a point where said photobiocatalyst is inactive, wherein said mixture comprises a volatile carbonaceous chemical; and
(b) an absorber comprising the absorbent medium, said absorber being in communication with said photo-biochemical reactor to receive the mixture and configured to separate out said volatile carbonaceous chemical from the mixture.

28. The system of claim 27, further comprising a photobioreactor for growing the photobiocatalyst.

29. The system of claim 27, further comprising a hydrogenation unit communicating with said photo-biochemical reactor to receive the volatile carbonaceous chemical.

30. The system of claim 27, wherein said absorbent medium is an isoparaffinic fluid.

31. The system of claim 30, wherein said isoparaffinic fluid is Isopar L™.

32. The system of claim 27, wherein said photo-biochemical reactor comprises a means for collecting said volatile carbonaceous chemical generated by said photobiocatalyst.

33. The system of claim 27, wherein said absorber comprises a means for isolating said volatile carbonaceous chemical generated by said photobiocatalyst.

34. The system of claim 33, wherein said means for isolating said volatile carbonaceous chemical comprises a separation tank.

35. The system of claim 27, wherein said photo-biochemical reactor comprises a means to handle the oxygen produced by photosynthesis, in a way that prevents the formation of an explosive mixture.

36. The system of claim 35, wherein said means for handling the oxygen is the free flow of the oxygen to the atmosphere.

37. The system of claim 35, wherein said means for handling the oxygen is a column to separate the aqueous, oil and gas phases by gravity.

38. The system of claim 28, wherein said photo-biochemical reactor and said photobioreactor are a single reactor.

39. The system of claim 27, further comprising a post-synthetic reactor unit to perform at least one or more post-synthetic modification steps, wherein said one or more post-synthetic modification steps are selected from reduction, hydrogenation, oxidation, oligomerization or polymerization to form homo- or hetero-oligomers or -polymers, esterification, hydrolysis, amination, carbonylation or decarbonylation.

40. The system of claim 29, wherein said hydrogenation unit comprises a means for hydrogenating said volatile carbonaceous chemical generated by said photobiocatalyst.

41. A hydrocarbon fuel produced by the method of claim 1.

42. A hydrocarbon fuel comprising isopentane or other hydrogenated products of photosynthetically produced isoprene or terpenes produced using a system comprising the steps of:
producing isoprene or other terpene from genetically modified cyanobacteria;
absorbing this isoprene or other terpene in an absorbent medium that is not essentially miscible with oxygen;
separating this isoprene or terpene from the absorbent medium; and
hydrogenation of the isoprene or terpene in the presence of hydrogen and a catalyst to produce the hydrocarbon fuel.
